(19) United States
(12) Patent Application Publication
(10) Pub. No.: US 2017/0079533 A1
(43) Pub. Date: Mar. 23, 2017

(54) Diabetes and Hypertension Screening by Assessment of Arterial Stiffness and Autonomic Function

(71) Applicant: Medici Technologies, LLC, Albuquerque, NM (US)

(72) Inventors: Mark Ries Robinson, Albuquerque, NM (US); Elena A. Allen, Albuquerque, NM (US)

(21) Appl. No.: 15/371,718

(22) Filed: Dec. 7, 2016

Related U.S. Application Data
(63) Continuation-in-part of application No. 14/470,927, filed on Aug. 27, 2014.
(60) Provisional application No. 62/263,833, filed on Dec. 7, 2015.
(60) Provisional application No. 61/987,476, filed on May 1, 2014.

(51) Int. Cl. A61B 5/02; A61B 5/00; A61B 5/021

(52) U.S. Cl. CPC A61B 5/02007; A61B 5/021; A61B 5/6814; A61B 5/6826; A61B 5/7275; A61B 5/02416

(57) ABSTRACT

The present invention provides methods and apparatuses to assess vascular stiffness of a subject, and to assess diabetes or hypertension from the assessment of vascular stiffness. Example embodiments comprise determining arrival at a peripheral site of a blood pressure wave as a function of time relative to the cardiac cycle of the subject at a plurality of measurement conditions, wherein at least two of the conditions are characterized by at least one of: (a) different central transmural pressure, (b) different peripheral transmural pressure; assessing vascular stiffness from the determinations at the plurality of measurement conditions.

### BACKGROUND OF INVENTION

Diabetes.

Diabetes mellitus is a major health problem in the United States and throughout the world's developed and developing nations. In 2002, the American Diabetes Association (ADA) estimated that 18.2 million Americans—fully 6.4% of the citizenry—were afflicted with some form of diabetes. Of these, 90-95% suffered from Type 2 diabetes, and 35%, or about 6 million individuals, were undiagnosed. See ADA Report, Diabetes Care, 2003. The World Health Organization (WHO) estimates that 175 million people worldwide suffer from diabetes, with Type 2 diabetes representing 90% of diagnoses. Unfortunately, projections indicate that this grim situation will worsen in the next two decades. The WHO forecasts that the total number of diabetics will double before the year 2025. Similarly, the ADA estimates that by 2020, 8.0% of the US population, some 25 million individuals, will have the disease. Assuming rates of detection remain static, this portends that in less than twenty years, three of every 100 Americans will be “silent” diabetics. It is no surprise that many have characterized the worldwide outbreak of diabetes as an epidemic.

Diabetes has a significant impact on individual health and the national economy. U.S. health care costs related to diabetes exceeded $132 billion in 2002. Due to the numerous complications that result from chronic hyperglycemia, these costs were distributed over a wide array of health services. For example, between 5 and 10 percent of all U.S. expenditures in the areas of cardiovascular disease, kidney disease, endocrine and metabolic complications, and ophthalmic disorders were attributable to diabetes. See ADA Report, Diabetes Care, 2003. These economic and health burdens belie the fact that most diabetes-related complications are preventable. The landmark Diabetes Control and Complications Trial (DCCT) established that a strict regimen of glucose monitoring, exercise, proper diet, and insulin therapy significantly reduced the progression of and risk for developing diabetic complications. See DCCT Research Group, N Eng J Med, 1993. Furthermore, the ongoing Diabetes Prevention Program (DPP) has already demonstrated that individuals at risk for diabetes can significantly reduce their chances of developing the disease by implementing lifestyle changes such a weight loss and increased physical activity. See DPP Research Group, N Eng J Med, 2002. The ADA has recommended that health care providers begin screening of individuals with one or more disease risk factors, observing: “If the DPP demonstrates a reduction in the incidence of Type 2 diabetes as a result of one or more of the [tested] interventions, then more widespread screening . . . may be justified”. See ADA Position Statement, Diabetes Care, 2003.

The Fasting Plasma Glucose (FPG) test is one of three accepted clinical standards for the diagnosis of or screening for diabetes. See ADA Committee Report, Diabetes Care, 2003. The FPG test is a carbohydrate metabolism test that measures plasma glucose levels after a 12-14 hour fast. Fasting stimulates the release of the hormone glucagon, which in turn raises plasma glucose levels. In non-diabetic individuals, the body will produce and process insulin to counteract the rise in glucose levels. In diabetic individuals, plasma glucose levels remain elevated. The ADA recommends administration of the FPG test in the morning because afternoon tests tend to produce lower readings. In most healthy individuals, FPG levels will fall between 70 and 100 mg/dl. Medications, exercise, and recent illnesses can impact the results of this test, so an appropriate medical history should be taken before it is performed. FPG levels of 126 mg/dl or higher indicate a need for a subsequent retest. If similarly, elevated levels are reached during the retest, a diagnosis of diabetes mellitus is typically made. Results that measure only slightly above the normal range may require further testing, including the Oral Glucose Tolerance Test (OGTT) or a postprandial plasma glucose test, to confirm a diabetes diagnosis. Other conditions that can cause an elevated result include pancreatitis, Cushing's syndrome, liver or kidney disease, eclampsia, and other acute illnesses such as sepsis or myocardial infarction.

The OGTT is the clinical gold standard for diagnosis of diabetes despite various drawbacks. After presenting in a fasting state, the patient is administered an oral dose of glucose solution (75 to 100 grams of dextrose) which typically causes blood glucose levels to rise in the first hour and return to baseline within three hours as the body produces insulin to normalize glucose levels. Blood glucose levels are typically be measured four to five times over a 3-hour OGTT administration. On average, levels typically peak at 160-180 mg/dl from 30 minutes to 1 hour after administration of the oral glucose dose, and then return to fasting levels of 140 mg/dl or less within two to three hours. Factors such as age, weight, and race can influence results, as can recent illnesses and certain medications. For example, older individuals will have an upper limit increase of 1 mg/dl in glucose tolerance for every year over age 50. Current ADA guidelines dictate a diagnosis of diabetes if the two-hour post-load blood glucose value is greater than 200 mg/dl on two separate OGTTs administered on different days.

Glycated Hemoglobin (hemoglobin A1c, A1c or HBA1c) is also recommended by the ADA for the diagnosis of or screening for diabetes, effective as of 2010. HBA1c is a form of hemoglobin that is influenced by the average plasma glucose concentration over the life of the red blood cell. It is formed in a non-enzymatic glycation pathway due to hemoglobin's exposure to plasma glucose. Normal levels of glucose produce a normal amount of glycated hemoglobin. As the average amount of plasma glucose increases, the fraction of glycated hemoglobin increases in a predictable way. This serves as a marker for average blood glucose levels over the months prior to the measurement, and therefore serves as a marker for diabetes. An HbA1c level greater than or equal to 6.6% is diagnostic of diabetes.

In addition to these diagnostic criteria, the ADA also recognizes two “pre-diabetic” conditions reflecting deviations from euglycemia that, while abnormal, are considered insufficient to merit a diagnosis of diabetes mellitus. An individual is said to have “pre-diabetes” when a single FPG test falls between 100 and 126 mg/dl or Hba1c is between 5.7 to 6.4%, or when the OGTT yields 2-hour post-load glucose values between 140 and 200 mg/dl. Both of these conditions are considered risk factors for diabetes. FIG. 1 is a visual representation of these screening criteria.

Pre-test fasting, invasive blood draws, and repeat testing on multiple days combine to make the OGTT, A1c and FPG tests inconvenient for the patient and expensive to administer. In addition, the diagnostic accuracy of these tests leaves significant room for improvement. See, e.g., M. P. Stern, et al., Ann Intern Med, 2002, and J. S. Yudkin et al., BMJ, 1990. Various attempts have been made in the past to avoid the disadvantages of the FPG and OGTT in diabetes screening. For example, risk assessments based on patient history and paper-and-pencil tests have been attempted, but such techniques have typically resulted in lackluster diagnostic accuracy.

A reliable, convenient, and cost-effective means to screen for diabetes mellitus is needed. Also, a reliable, convenient, and cost-effective means for measuring effects of diabetes could help in treating the disease and avoiding complications from the disease.

Hypertension is defined as a physician office systolic blood pressure (BP) of ≧140 mmHg and diastolic BP of ≧90 mmHg. Normal blood pressure is defined a systolic BP<120 mmHg and diastolic BP<80 mmHg. The gray area between systolic BP of 120-139 mmHg and diastolic BP of 80-89 mmHg is defined as “pre-hypertension.” Despite these simple criteria, accurate determination of hypertension is difficult due to the fact that a point measurement of blood pressure might not reflect true ambulatory blood pressure. Patients with white coat hypertension (WCH) can be especially problematic. Patients with WCH have an elevated office BP and normal home BP measurements or ambulatory blood pressure monitoring. The prevalence of WCH in the general population has been reported to be 20%. The presence of WCH is also problematic in diabetics: a recent large study found WCH in 33% of diabetic patients (Gorostidi M, de la Sierra A, Gonzalez-Albarran O, et al.; Spanish Society of Hypertension ABPM Registry investigators. Abnormalities in ambulatory blood pressure monitoring in hypertensive patients with diabetes. Hypertens Res 2011; 34: 1185-1189). Subjects with WCH may receive long-term drug treatment that is both unnecessary and expensive. Currently, the only way to prevent over-diagnosis of hypertension is to confirm it by 24-h ambulatory BP monitoring, which is itself cumbersome, expensive and device dependent. Thus, a simple test that can identify WCH would have significant value in the practice of medicine.

Arterial Compliance.

The classic definition by Spencer and Denison of compliance (C) is the change in arterial blood volume (ΔV) due to a given change in arterial blood pressure (ΔP). So, C=ΔV/ΔP. Arterial compliance provides an index of the elasticity of large arteries. Arterial compliance is an important cardiovascular risk factor. Compliance generally diminishes with age. Age affects the wall properties of central elastic arteries (aorta, carotid, iliac) in a different manner than in muscular arteries (brachial, radial, femoral, popliteal). With increasing age, the pulsatile strain breaks the elastic fibers, which are replaced by collagen. On the other hand, there is only little alteration of compliance in the muscular, i.e. distal, arteries with age.

Pulse pressure waves, generated by the left ventricle, travel through the arterial tree and are reflected at multiple peripheral sites. As a result, the arterial pressure waveform at any site is a combination of the forward travelling waveform and the backward (or reflection) waveform. In individuals with healthy and compliant arteries, the two waveforms merge during diastole and augment coronary perfusion. With aging, the arterial wall thickens and the arteries get stiffer. As a result, the pressure waves travel faster and the reflected pressure wave returns during the systolic phase, increasing systolic pressure and thus increasing left ventricular load.

The most common method for determining arterial compliance is the measurement of Pulse Wave Velocity (PWV). In cardiovascular research and clinical practice, PWV refers to the velocity of pressure pulses that propagate along the arterial tree due to left ventricular ejection. At the opening of the aortic valve, the sudden rise of aortic pressure is absorbed by the elastic aorta walls. Subsequently, a pulse wave naturally propagates along the aorta exchanging energy between the aortic wall and the aortic blood flowError! Reference source not found. It is important to note that PWV is influenced by both arterial stiffness and the blood pressure in the vessel.

Modifications of the arterial wall compliance or stiffness will induce changes in the velocity at which pressure pulses travel in the artery. The Bramwell and Hill equation defines the relationship between PWV and the compliance of the artery:

$$PWV = \sqrt{\frac{V}{ρ C}}$$

The Bramwell and Hill equation states that PWV is inversely proportional to the square root of the vessel compliance, at given arterial volume, V, and blood density, ρ, assuming that the artery wall is isotropic and experiences isovolumetric change with pulse pressure.

The determination of aortic PWV is considered to be the gold standard of arterial stiffness measurements. Aortic PWV is a measure of the speed of the arterial pressure waves travelling along the aortic and aorto-iliac pathway. Higher arterial pressure wave velocity is indicative of stiffer arteries. FIG. 2 is an illustration of aortic PWV, which is defined as the average velocity of a pressure pulse when travelling from the aortic valve, through the aortic arc until it reaches the iliac bifurcation. PTT is the Pulse Transit Time.

Autonomic Function.

The autonomic nervous system is a division of the peripheral nervous system that controls automated body functions including heart rate, blood pressure, digestion and metabolism. The autonomic nervous system is divided into parasympathetic and sympathetic components, which work antagonistically to provide a very fine degree of control over their target organs. In general, the parasympathetic nervous system predominates during rest by slowing heart rate, lowering blood pressure, and promoting digestion. The sympathetic nervous system is recognized for mounting responses to physical and psychological stimuli. Autonomic function is most often estimated noninvasively by measuring heart rate variability. Heart rate variability refers to the beat-to-beat variability of heart rate measured over a period of time. The heart rate of a healthy heart is not fixed but rather varies over milliseconds in response to moment-to-moment physiological changes. Low heart rate variability generally reflects poor autonomic tone. Autonomic dysfunction, or improper autonomic responsiveness to challenge, is correlated with a number of adverse health behaviors and diseases. Diabetes and hypertension are the most commonly associated with autonomic dysfunction. In individuals with diabetes, prolonged hyperglycemia leads to degradation of the microvasculature, leading to a specific form of autonomic dysfunction term “diabetic autonomic neuropathy”.

### SUMMARY OF INVENTION

Embodiments of the present invention provide a reliable, convenient, and cost-effective means to screen for diabetes mellitus and hypertension. The diabetes and hypertension assessment system is composed of a simple noninvasive PPG-based technique for measuring in vivo the arterial distensibility over a range of pressures. Changes in arterial pressure are generated via changes in hydrostatic pressure or stroke volume during simultaneous measurement of pulse transit times. Pulse transit times are converted into pulse wave velocities, which have a direct association with arterial distensibility. The determination of pulse wave velocity over a range of transmural pressures creates an arterial compliance curve that can be used to determine the likelihood of diabetes or hypertension. This application is related to U.S. provisional application 62/263,833, filed Dec. 7, 2015, and to U.S. utility application Ser. No. 14/470,927, filed Aug. 27, 2014, and to U.S. provisional application 61/987,476, filed May 1, 2014, each of which is incorporated herein by reference.

### BRIEF DESCRIPTION OF THE DRAWINGS

FIG. 1 is a schematic illustration of diabetes screening criteria.

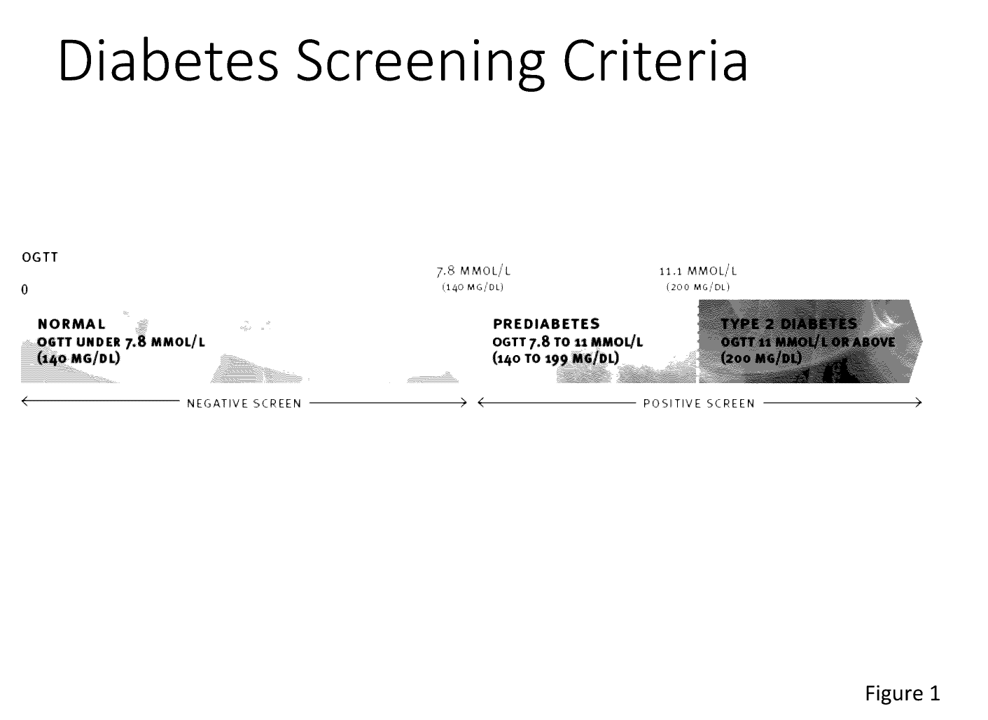

FIG. 2 is a schematic illustration of aortic pulse wave velocity.

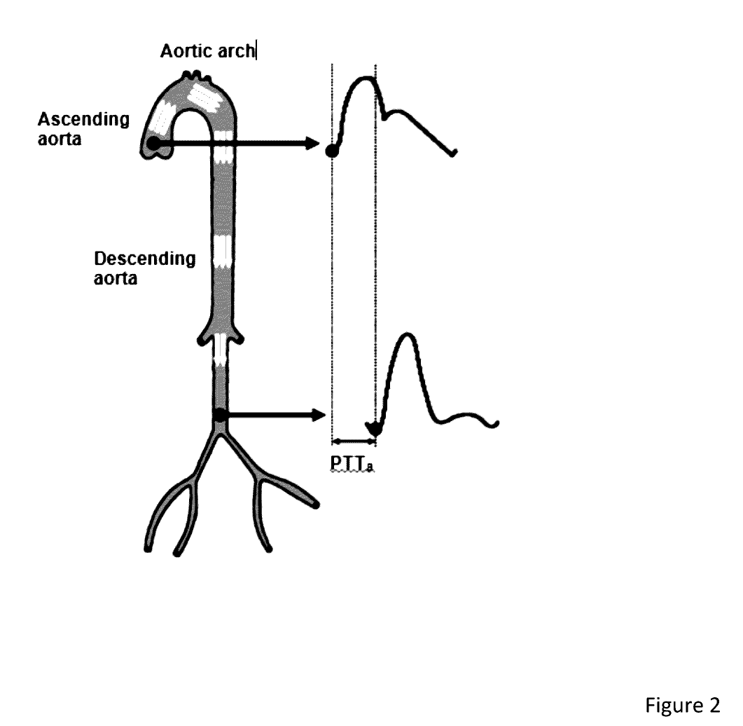

FIG. 3 is a schematic illustration of type II diabetes progression.

FIG. 4 is schematic illustration of parameters measured by the current invention.

FIG. 5 is a schematic illustration of the results of analysis of three different measures of compliance in three groups with different glucose control.

FIG. 6 is a schematic illustration of the relationship between pressure and pulse wave velocity.

FIG. 7 is a schematic illustration of the nonlinear relationship between pressure and cross-sectional area.

FIG. 8 is a table of heart rate variability results in diabetics and controls.

FIG. 9 is a schematic depiction of the relationship between pulse transit time and arm transit time.

FIG. 10 is a schematic illustration of a method for the calculation of augmentation index.

FIG. 11 is a schematic illustration of arm positions useful for peripheral compliance determination.

FIG. 12 is a representation of pulse data from arm at 0°.

FIG. 13 is a representation of pulse data from arm at 45°.

FIG. 14 is a representation of pulse data from arm at 90°.

FIG. 15 is a representation of Pulse data from arm at 135°.

FIG. 16 is a representation of pulse data from arm at 180°.

FIG. 17 is a representation of Measured Pulse Transit Time and Pulse Wave Velocity.

FIG. 18 is a representation of a Calculated distensibility curve.

FIG. 19 is a plot of PAT during an arm swing test

FIG. 20 is illustrates autonomic changes in the terminal capillary.

FIG. 21 is a plot of wrist and finger PPG data during an arm swing.

FIG. 22 is a plot of pulse wave velocity versus arm transmural pressure.

FIG. 23 is an illustration of stroke volume change as a function of controlled resistance breathing

FIG. 24 is a representation of variable resistance breathing.

FIG. 25 is a representation of variable exhalation resistance resulting in stroke volume variance.

FIG. 26 is a representation of a physiological changes due to resistance breathing.

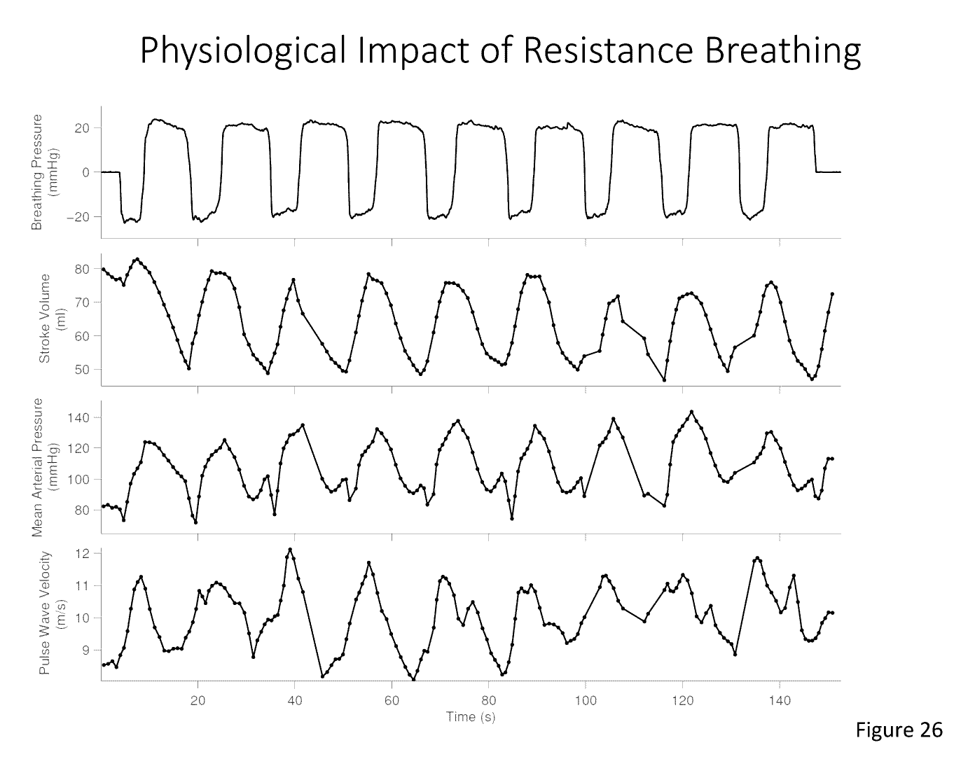

FIG. 27 is a plot of relationship between transmural pressure and pulse wave velocity.

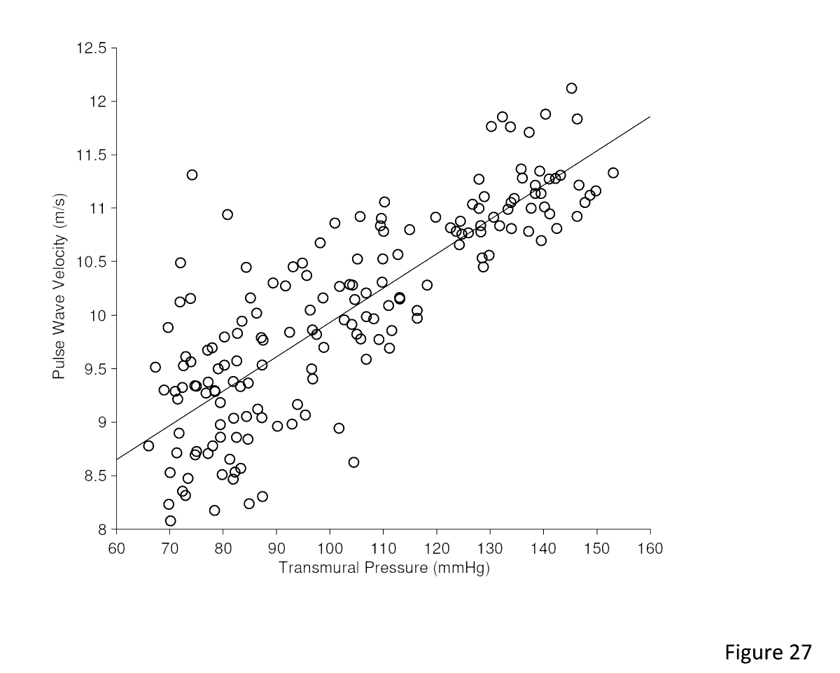

FIG. 28 is a schematic illustration of an example of distensibility versus pressure with age differences.

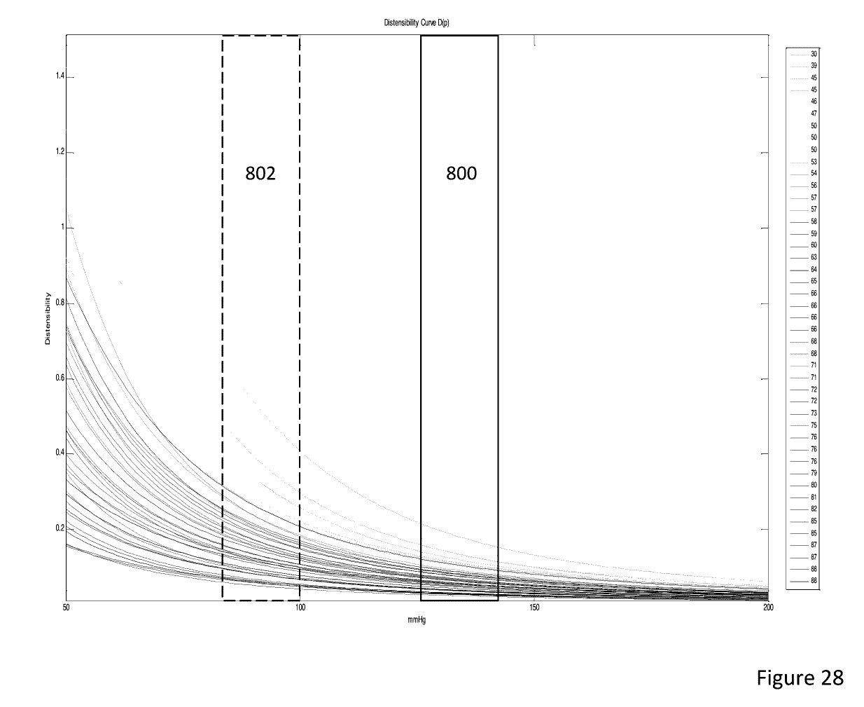

FIG. 29 is a plot illustrating the impact of Valsalva maneuver on blood pressure.

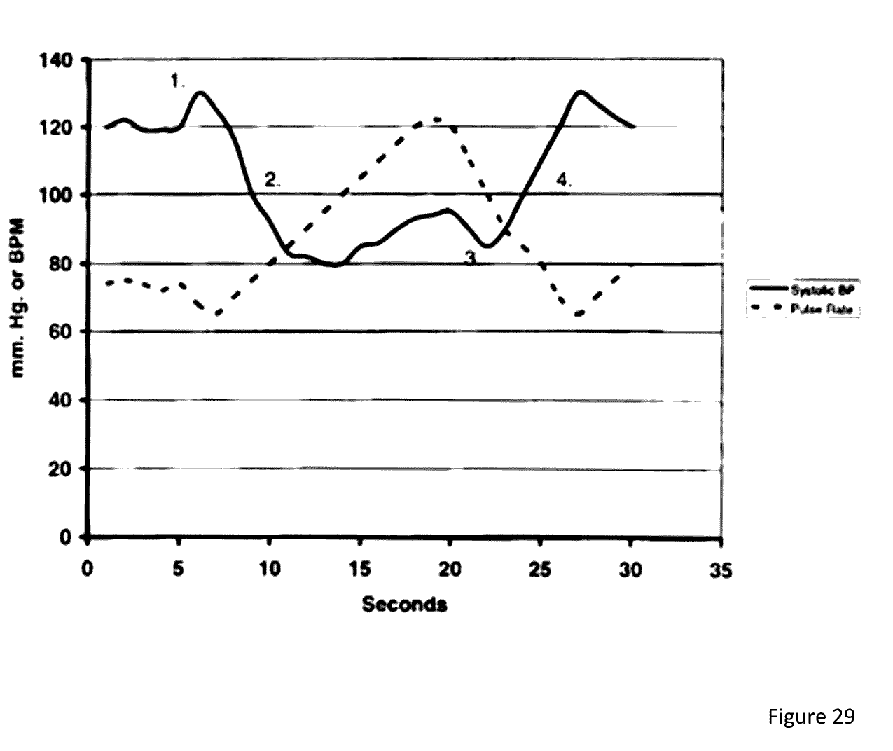

FIG. 30 is a plot illustrating pulse wave velocity as a function of age.

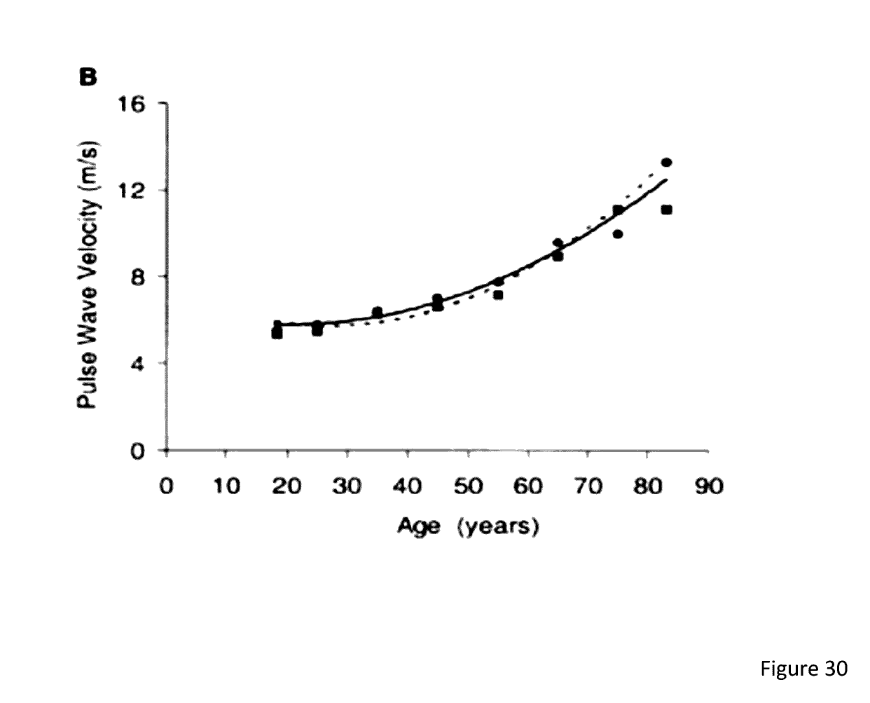

FIG. 31 is an illustration of the calculation of augmentation index.

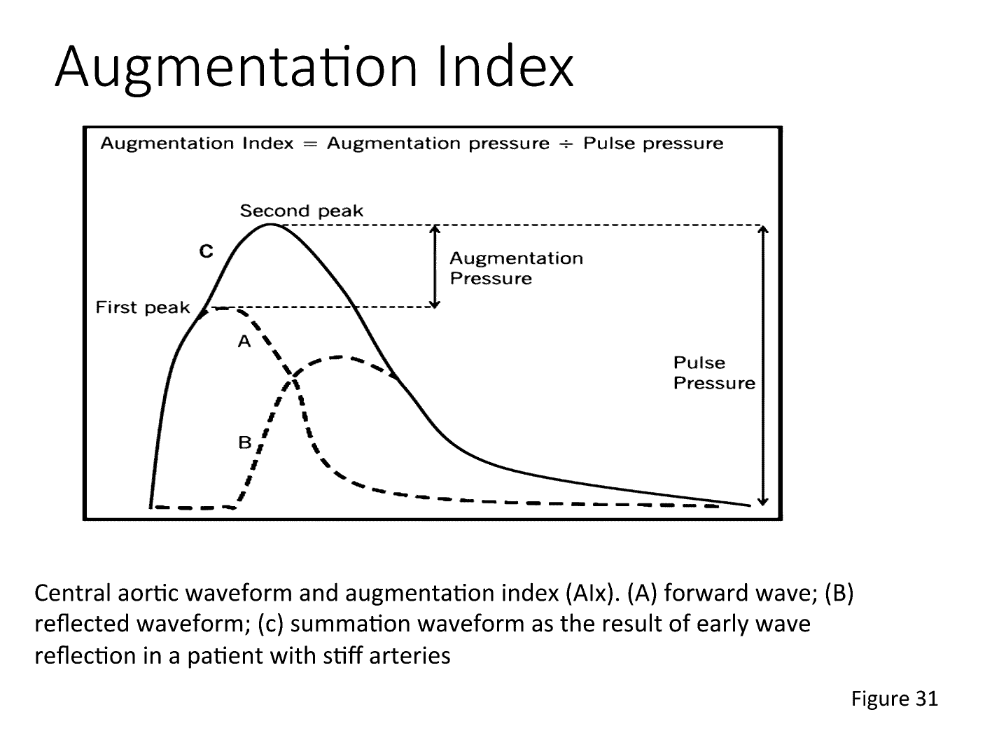

FIG. 32 is a plot illustrating post contour variation is a function of age.

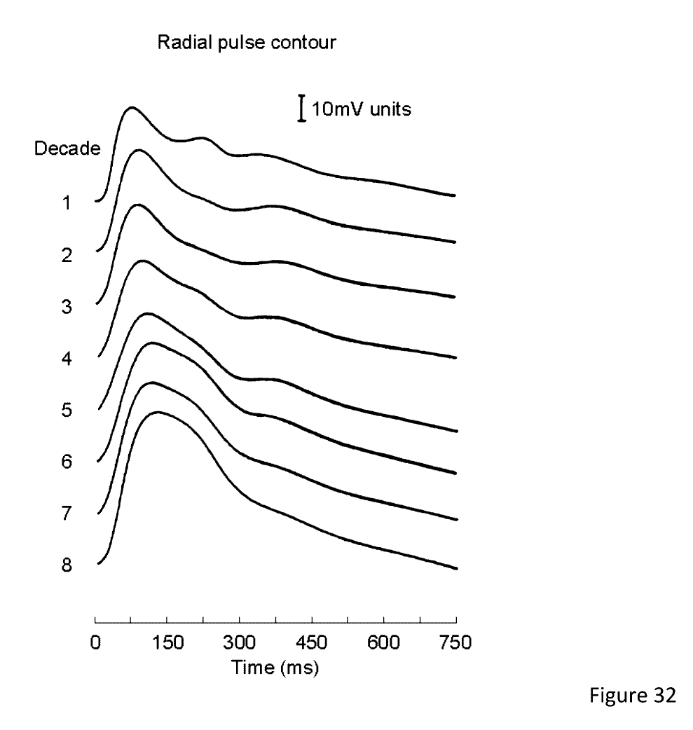

FIG. 33 is an illustration of example pulse waveform data.

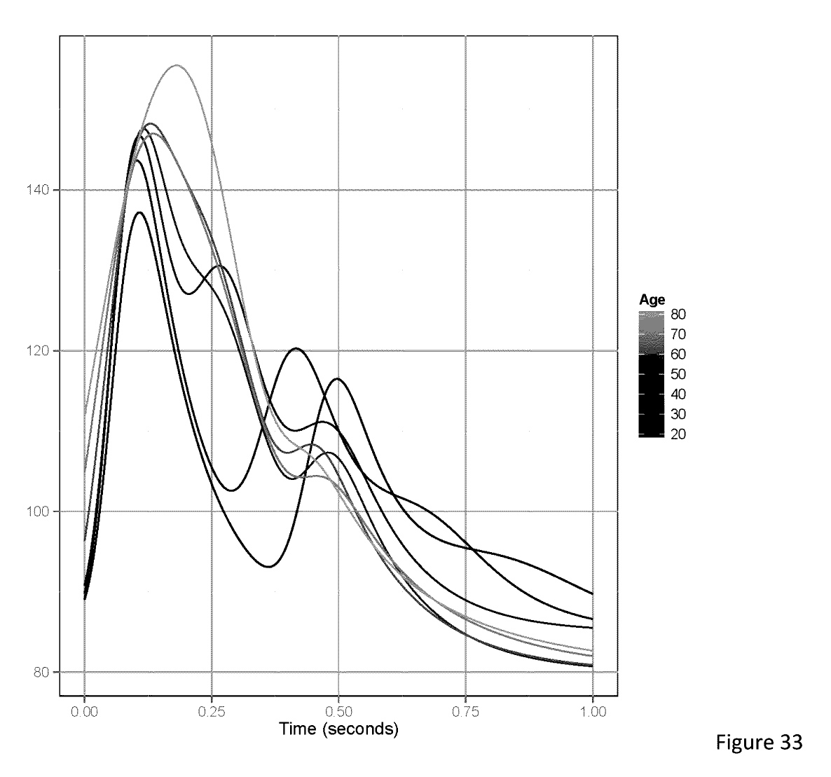

FIG. 34 is a receiver operator characteristic curve showing improved classification capability.

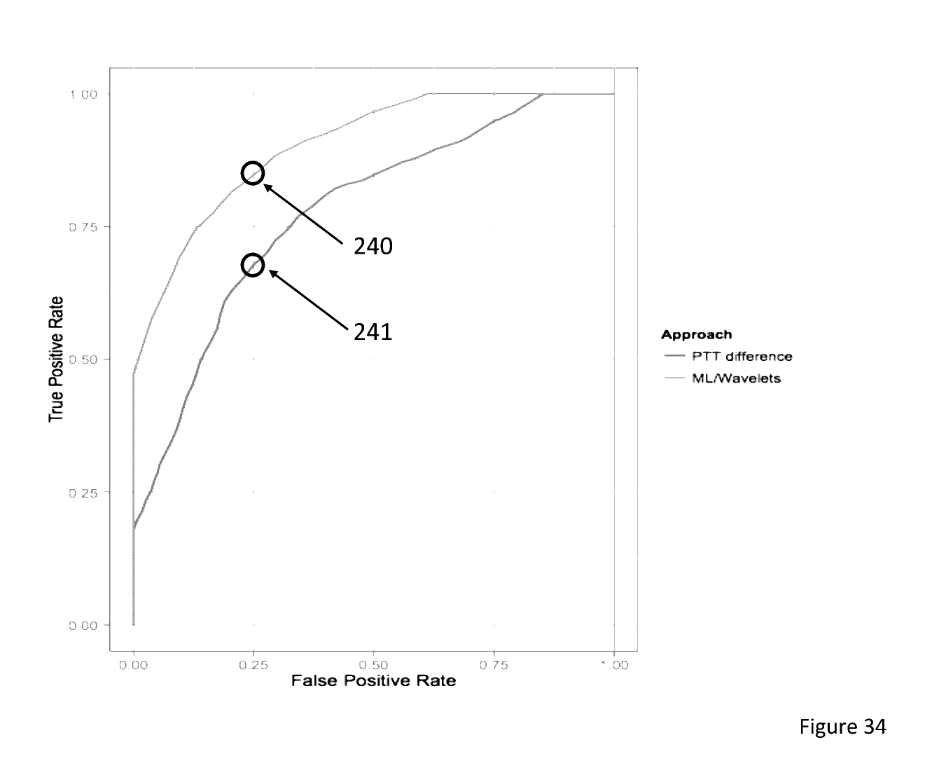

FIG. 35 is a schematic illustration of an example of screening device.

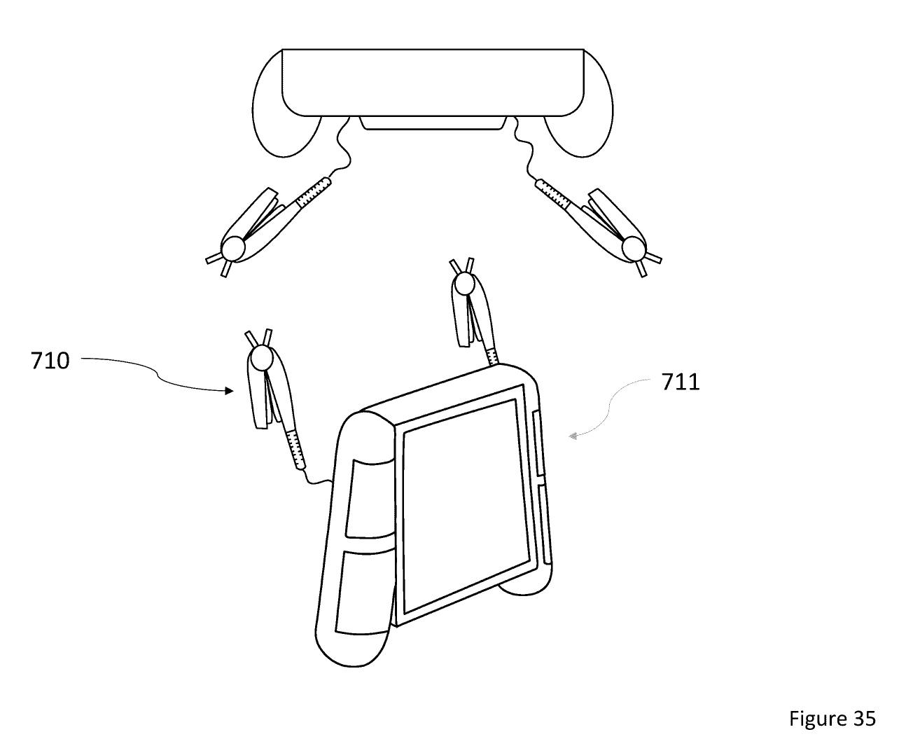

FIG. 36 is a schematic illustration of an example display of screening system.

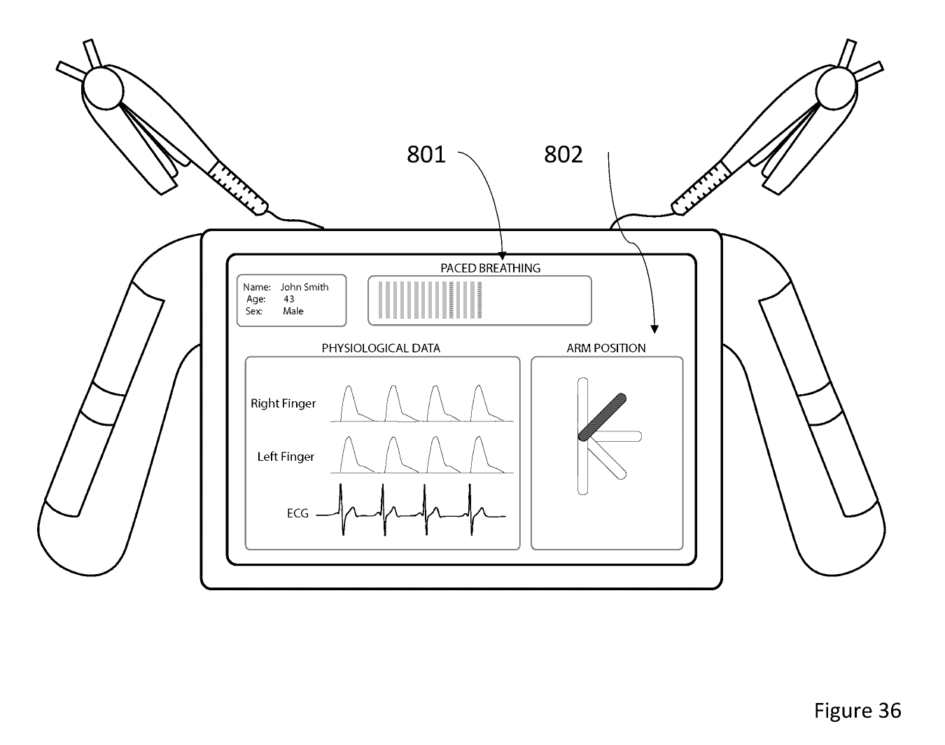

FIG. 37 is a schematic illustration of an example of screening test.

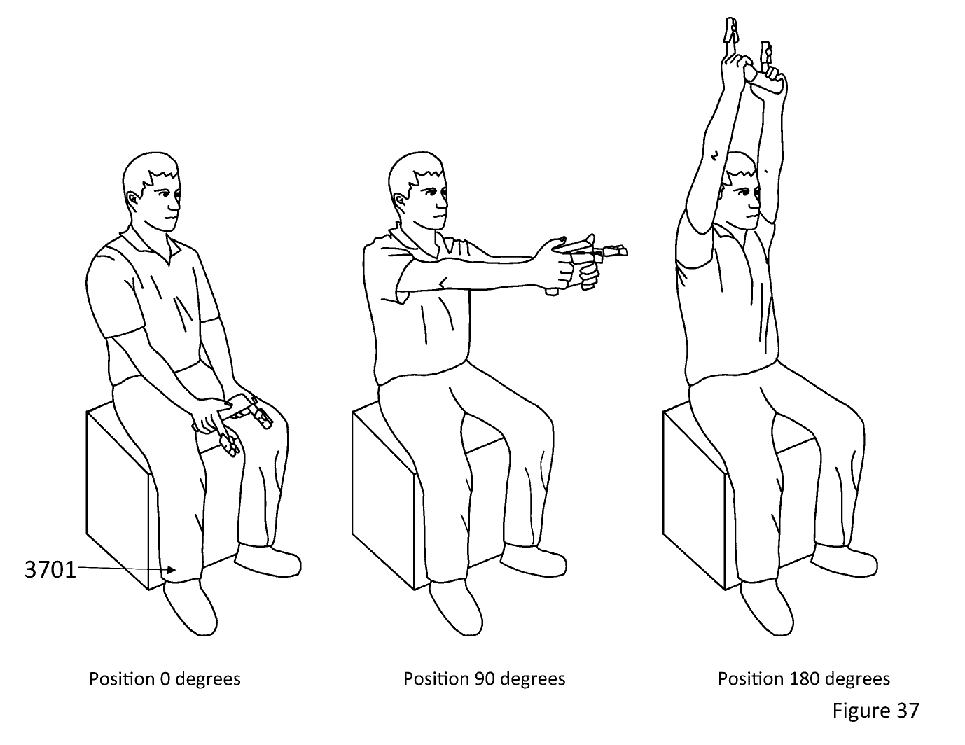

FIG. 38 is a schematic illustration of an example of resistance breathing.

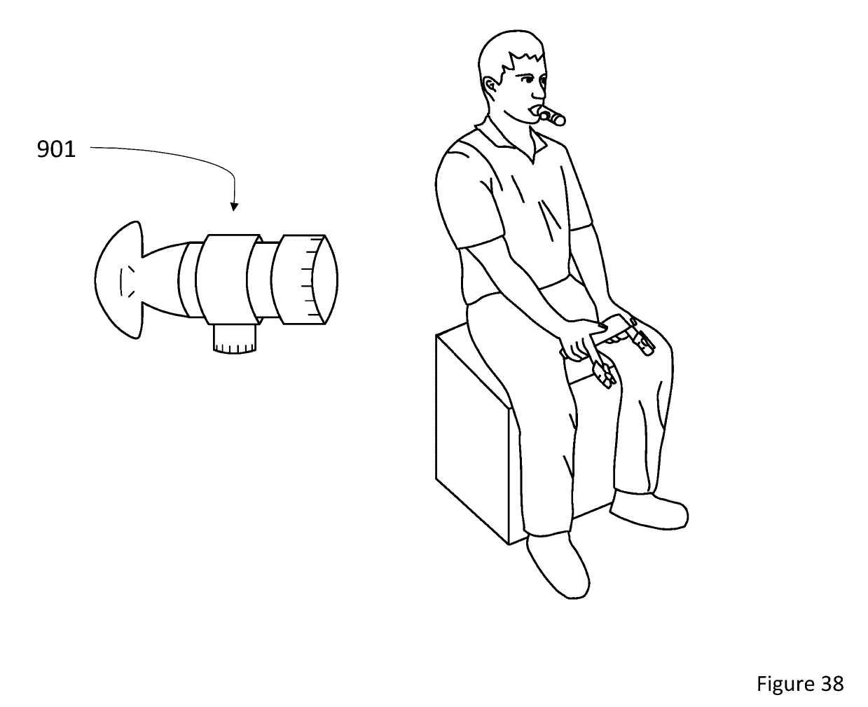

FIG. 39 is a schematic illustration of an example of a screening test without blood pressure.

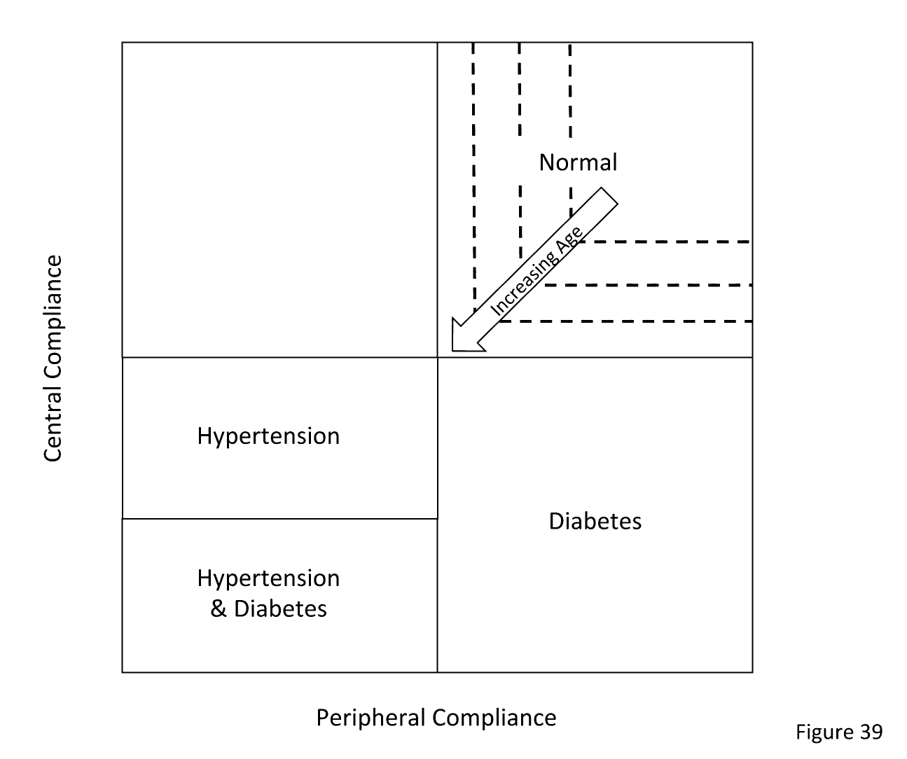

FIG. 40 is a schematic illustration of an example of information utilization with blood pressure included.

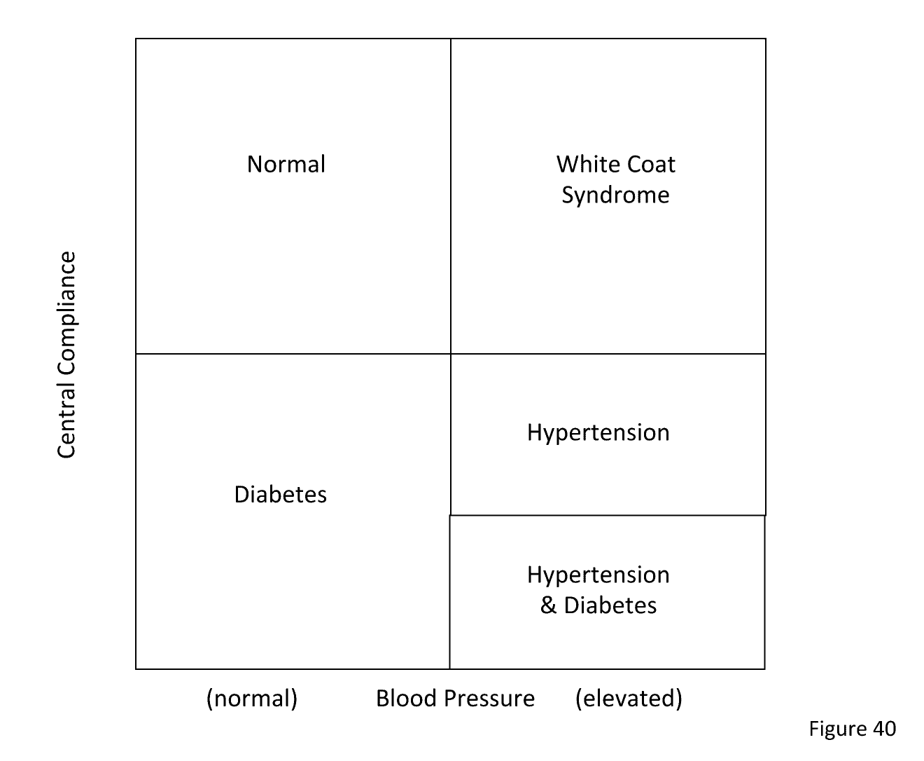

FIG. 41 is a schematic illustration of an example embodiment of screening system.

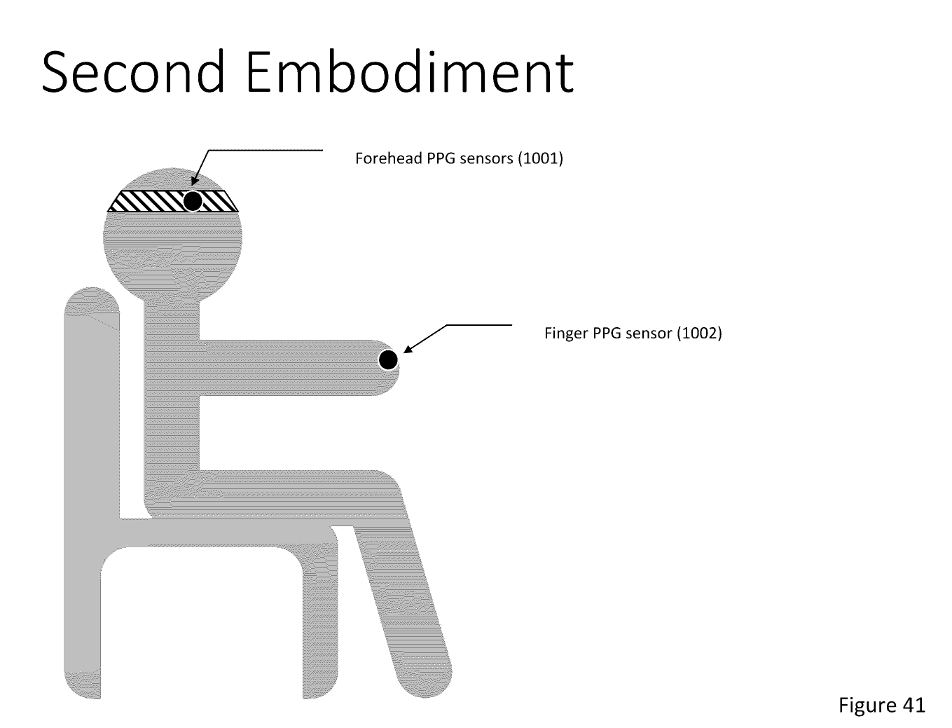

### DESCRIPTION OF THE INVENTION

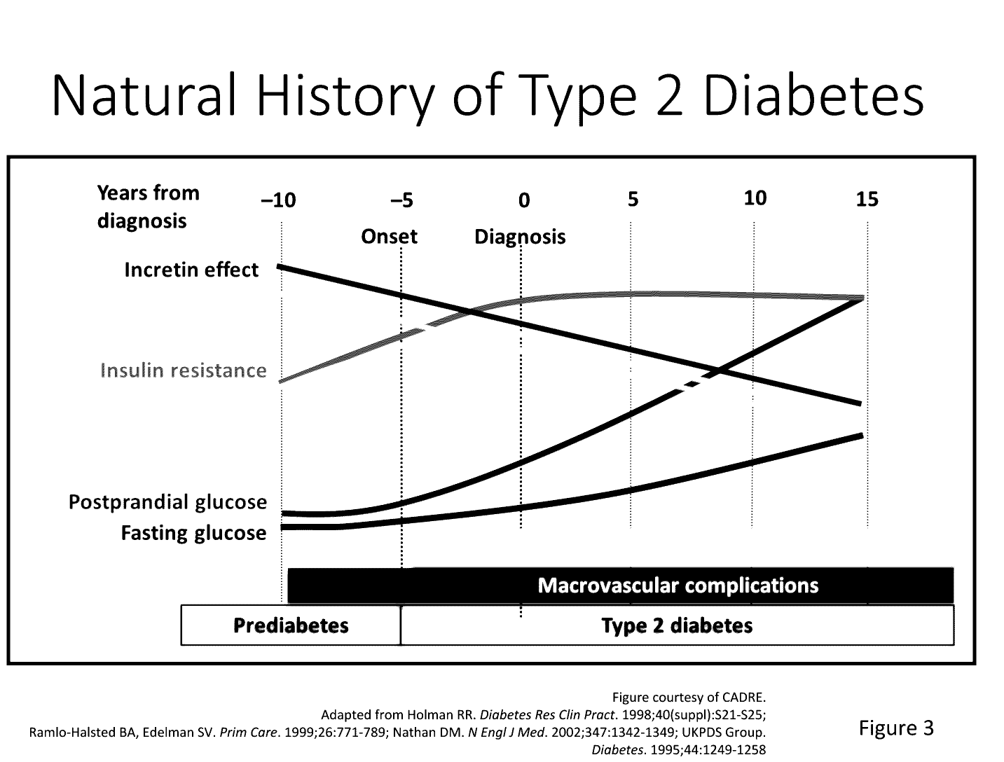

Current diabetes testing methods are based upon the body's inability to control glucose either during a fasting state or after being subjected to a glucose load. However, the true pathophysiology of diabetes contains a number of other physiological markers that are predictive of prediabetes and diabetes. The initiation of diabetes begins with an increase in insulin resistance and impairments in β-cell function. Over time, relative insulin deficiency occurs as well as excessive glucagon production leading to overproduction of endogenous glucose in the liver. These malfunctions in glucose control eventually lead to postprandial hyperglycemia and then elevations in fasting blood glucose levels. These relationships are shown graphically in FIG. 3, obtained from the American Association of Clinical Endocrinologists Diabetes Research Center, Adapted from Holman R R. Diabetes Res Clin Pract. 1998; 40(suppl):S21-S25; Ramlo-Halsted B A, Edelman S V. Prim Care. 1999; 26:771-789; Nathan D M. N Engl J Med. 2002; 347:1342-1349; UKPDS Group. Diabetes. 1995; 44:1249-1258.

In addition to these changes in the ability to regulate glucose, additional changes occur with respect to the vascular system and autonomic nervous system. Examination of FIG. 3 shows that macrovascular changes as well as microvascular changes occur prior to typical diagnosis. It is especially important to note that macrovascular changes occur very early in the natural progression of Type II diabetes. As it relates to diabetes assessment, the identification of these macrovascular changes can create a diabetes assessment test that can identify the disease earlier and result in improved sensitivity.

Embodiments of the present invention provide an ability to detect vascular changes associated with prediabetes, diabetes and hypertension by examination of arterial stiffness. Embodiments of the invention relate to the determination of arterial stiffness as a method for diabetes and hypertension assessment based upon changes in transmural pressure. The information provided by vascular assessment can be combined with information associated with autonomic function for an improved diabetes assessment screening that is noninvasive and simple to use. The same information can be used to create an improved hypertension test with the specific ability to determine the presence of white coat hypertension.

Embodiments of the current invention enable a diabetes and hypertension assessment system composed of a simple noninvasive PPG-based technique for measuring in vivo the arterial distensibility over a range of pressures. Changes in arterial pressure are generated via changes in hydrostatic pressure or stroke volume during simultaneous measurement of pulse transit times. Pulse transit times are converted into pulse wave velocities, which have a direct association with arterial distensibility. The determination of pulse wave velocity over a range of transmural pressures creates an arterial compliance curve that can be used to determine the likelihood of diabetes or hypertension.

### DEFINITIONS

As used herein, “diabetes assessment” includes determining the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes. The term “diabetes” includes a number of blood glucose regulation conditions, including Type I, Type II, and gestational diabetes, other types of diabetes as recognized by the American Diabetes Association (See ADA Committee Report, Diabetes Care, 2003), hyperglycemia, impaired fasting glucose, impaired glucose tolerance, and pre-diabetes.

As used herein, photoplethysmography (PPG) is an optical measurement technique that can be used to detect blood volume changes in tissue or has a signal that is related to the cardiac cycle. A PPG can be used to create a digital volume pulse, and these terms are often used interchangeably. Absorption of light by red blood cells gives a digital volume pulse (DVP), which can be acquired continuously without much medical training. The amplitude of the DVP is determined by local blood perfusion, but its contour is determined by characteristics of the whole systemic circulation. For the purposes of this application, PPG and DVP may be used interchangeably to describe the signal acquired.

Arterial compliance refers to the general ability of a blood vessel wall to expand and contract passively with changes in pressure and includes a multitude of metrics and terms used to refer to related properties such a stiffness, elastance, Young's modulus, elastic modulus, distensibility, and other parameters.

Arterial compliance function is a function, typically defined by multiple parameters, that defines the relationship between increasing volume with increasing transmural pressure, or the tendency of a hollow organ to resist recoil toward its original dimensions on application of a distending or compressing force. The arterial compliance function is a relationship that defines a continuous function describing the physiological response of the vessel.

Arterial “compliance-state” is a measurement of compliance that defines the “compliance-state” under given measurement conditions. A vessel will have inherent compliance properties as defined by the compliance function but any single compliance measurement of a vessel is the combination of the vessels properties and the state or condition of the vessel during the measurement. Specifically, pressure does not change the arterial compliance function, but blood pressure will impact the measured arterial “compliance-state”.

Autonomic function refers to the functional characteristics of the autonomic nervous system (ANS), a division of the peripheral nervous system that influences the function of internal organs. The autonomic nervous system is a control system that acts largely unconsciously and regulates bodily functions such as the heart rate, digestion, respiratory rate, pupillary response, urination, and sexual arousal.

Transmural pressure is a general term for pressure across the wall of an object of vessel (transmural means “across the wall”) and is defined by the following equation:

P_{TM}=P_{Inside}−P_{Outside}

A flexible container or object expands if there is a positive transmural pressure (pressure greater inside than outside the object) and contracts with a negative transmural pressure. A positive transmural pressure is sometimes referred to as a “distending” pressure. Changes in transmural pressure influence the arterial “compliance-state” of the vessel and pulse wave velocity. For example, increasing systemic blood pressure does not change the arterial compliance function but it will affect the measure “compliance-state”. The artery will increase in diameter and decrease in thickness. The increase in diameter will result in the recruitment of collagen fibers, which will increase the stiffness of the vessel under these measurement conditions. Hence, the “compliance-state” of the arterial wall will depict a strong dependence on transmural pressure. Transmural pressure changes refer to any mechanism that changes the relationship between inside pressure and outside pressure. Methods for changing inside or intravascular pressure include but are not limited to positional changes, hydrostatic pressure changes, stroke volume changes, volume changes, cardiac contractility changes, and exercise. Methods for changing outside or extra vascular pressure include but are not limited to changes in intrathoracic pressure, positional changes, compression of the vasculature by water, air or other means, use of vacuum methodologies, resistance breathing, mechanical breathing, abdominal compression, Valsalva, Mueller maneuvers, and muscle contraction.

Resistance breathing is a general term that applies to any method that increases, decreases, or changes intrathoracic pressure over normal breathing. Resistance breathing tests can include inhalation resistance breathing, and exhalation resistance breathing, independently or in combination. The use of exhalation resistance breathing will create an increase in intrathoracic pressure while the use of inhalation resistance breathing creates decreased intrathoracic pressures. Additionally, a system may require different levels of resistance over the course of the protocol. A system can create and monitor if needed the inspiratory pressure and expiratory pressure of the subject so that highly repeatable results are obtained. Resistance breathing can be conducted using various protocols. For example, protocols may use paced breathing, which comprises target times for inhalation and exhalation such that the breathing rate is constant. Alternatively, event breathing is a type of resistance breathing where the subject exhales or inhales against resistance for a single breath followed by rest or recovery period. The event duration can be as long as 30 seconds, as an example. This type of event resistance breathing has advantages from a patient perspective in terms of ease of implementation and allows the subject to return to more of a pre-test condition with each activity. Additionally, the term resistance breathing covers the process of creating a change in intrathoracic pressure where little or no air movement occurs. The creation of an occlusion pressure either increased or decreased is encompassed as part of the broad definition of resistance breathing.

Controlled breathing is respiration where the rate of respiration, depth of respiration and the flow rate are controlled to the extent possible. Controlled breathing can be modified for subject size and can be used to further control respiration during testing. Controlled breathing can be used with resistance breathing to improve the repeatability of the test.

Hydrostatic positional change is a general term that applies to any process that changes the hydrostatic pressure in a vessel due to positional changes.

The terms compliance, stiffness and distensbility are related terms associated with the relationship between increasing volume with increasing pressure. These terms may be used interchangeably to describe this relationship.

### ASPECTS OF THE INVENTION

The invention provides methods and apparatuses for the assessment of diabetes and hypertensive status. The parameters utilized for the assessment involve changes in transmural pressure for the creation of a compliance assessment. The following paragraphs will provide information regarding (1) measurement systems used to obtain physiological data from the patient, (2) how the physiological data can be processed to obtain relevant physiological metrics, (3) what perturbations can be used for hemodynamic assessment, and (4) what metrics can be determined and reported to the care provider or patient.

Measurement Systems

The determination of vascular compliance requires the measurement of several physiological parameters. A brief description of these measurements systems is included herein.

Electrocardiography.

The function of the cardiovascular system can be monitored by a variety of methods. Electrocardiography (ECG or EKG*) is the process of recording the electrical activity of the heart over a period of time. Historically, the processes used electrodes placed on the skin, but newer devices no longer use electrodes. The sensors detect the tiny electrical changes on the skin that arise from the heart muscle's electrophysiologic pattern of depolarizing during each heartbeat. Phonocardiography (PCG) is a method of detecting the sounds produced by the heart and blood flow. Similar to auscultation, PCG is most commonly measured noninvasively from the chest with a microphone. Ballistocardiography (BCG) and seismocardiography (SCG) are both methods for studying the mechanical vibrations that coupled to the body and are produced by the cardiovascular system. BCG is a method where the cardiac reaction forces acting on the body are measured. SCG, on the other hand, is a method where the local vibrations of the precordium are measured.

Pulse Measurement.

A pulse measurement device is a system that enables the measurement of a pulse due to ejection of blood by the heart. A number of methods and systems can be used and the following is a list of some common approaches. Photoplethysmography (PPG) is an optical measurement technique that can be used to detect blood volume changes in tissue or has a signal that is related to the cardiac cycle. In addition to the PPG based methods, laser Doppler probes, tonometers and pulse transducers can be used to acquire signals related to the cardiac cycle. Typical pulse transducers use a piezo-electric element to convert force applied to the active surface of the transducer into an electrical analog signal that is related to the cardiac cycle.

Noncontact pulse detection methods have been developed over the past several years and enable pulse determination based upon image analysis. An example of a suitable procedure for remote PPG measure can follow the steps as proposed in McDuet et al. (2014), “Remote Detection of Photoplethysmographic Systolic and Diastolic Peaks Using a Digital Camera”. Additional information on the method is available in the article by Li, Xiaobai, et al. “Remote heart rate measurement from face videos under realistic situations” Proceedings of the IEEE Conference on Computer Vision and Pattern Recognition. 2014, which describes a system that can compensate for subject movement and changes in ambient light conditions. These noncontact systems can be used to enhance usability of the system.

Pulse measurement can also be done using the electro-pneumatic vascular unloading technique based upon the principals originally developed by Czech physiologist Jan Pe\check{n}áz. The systems measure blood pressure via combined pneumatic pressure system and an optical system. Blood volume changes caused by the pulsation of the blood in the artery (heart activity) are detected by infrared sensors. Counter pressure is exerted from the outside against the finger in such a way that the arterial wall is totally unloaded. This continuously changing outside pressure keeps the arterial blood volume constant all the time and directly corresponds to the arterial pressure. The intra-arterial pressure is therefore measured indirectly. The system represents an alternative method to measuring pulses. The current invention can use a combination of the above to create a unique monitoring system.

Measured Parameters

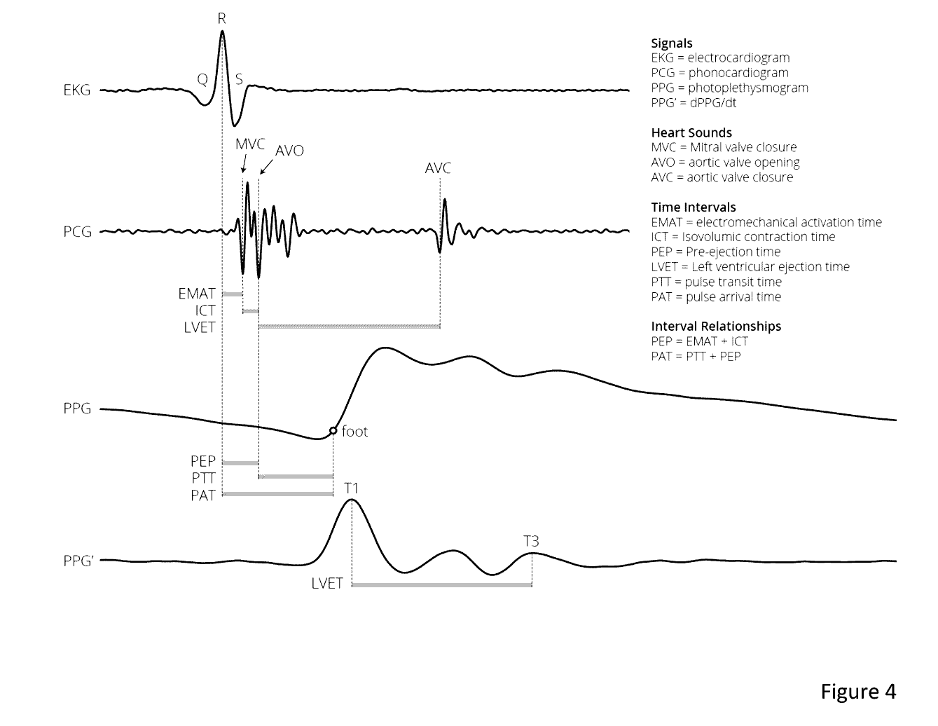

FIG. 4 shows the relationships between certain measured parameters and serves a reference for future terminology.

PAT.

The pulse arrival time (PAT) indicates the time from the onset of ventricular depolarization to the arrival of the pulse wave at a peripheral recording site, such as the finger or the forehead. The onset of ventricular depolarization is defined as the first negative deflection (Q wave) in the QRS complex as recorded with an electrocardiogram. However, in practice, this point is often identified as the positive deflection (R peak) in the QRS complex because the R wave is larger and therefore easier to detect. The arrival of the pulse wave in the periphery is measured by PPG and is defined by the “foot” of the wave. Following the method of Gaddum et al., the foot is determined as the intersection between (1) a horizontal projection through a local minimum preceding the wave arrival and (2) a projection through the subsequent local maximal gradient (slope) associated with the pulse wave. Gaddum, N. R., et al. “A technical assessment of pulse wave velocity algorithms applied to non-invasive arterial waveforms.” Annals of biomedical engineering 41.12 (2013): 2617-2629. The PAT is decomposed into the pulse travel time (PTT) and pre-ejection period (PEP), according to the following equation: PAT=PTT+PEP. The time intervals PEP and PTT are described below.

PEP.

The pre-ejection period (PEP) defines the time interval from the onset of ventricular depolarization to the opening of the aortic valve (i.e., beginning of ventricular ejection). It comprises both the electromechanical activation time (EMAT) and isovolumic contraction time (ICT). The onset of ventricular depolarization is defined as the ECG R wave, as described above, and the opening or the aortic valve is determined from the first heart sound (S1) recorded by PCG. Because aortic valve opening (AVO) lacks a distinct phonological signature in S1, we adopt the method of Paiva et al. and identify AVO using a Bayesian approach. Priors for AVO include (1) a local minimum in the PCG signal during 51, (2) large instantaneous amplitude as determined using the Hilbert Transform, and (3) a Gaussian distribution centered 30 ms after the closure of the mitral valve, which corresponds to the first negative deflection in S1. Paiva, R. P., et al. “Assessing PEP and LVET from heart sounds: algorithms and evaluation.” 2009 Annual International Conference of the IEEE Engineering in Medicine and Biology Society. IEEE, 2009. Note that PEP may also be defined as PEP=EMS−LVET, where EMS is electromechanical systole (the time interval from ventricular depolarization to the closure of the aortic valve) and LVET is the left ventricular ejection time. This approach is discussed below.

The PEP is a systolic time interval (STI) that allows assessment of ventricular function. As reviewed by Lewis et al., PEP is prolonged when preload decreases and is shortened when preload increases. Lewis, Richard P., et al. “A critical review of the systolic time intervals.” Circulation 56.2 (1977): 146-158. Although PEP additionally depends on afterload, and contractility, work by Bendjelid et al. has demonstrated in deeply sedated, mechanically ventilated patients that PEP is predominantly influenced by changes in ventricular preload. Bendjelid, Karim, Peter M. Suter, and Jacques A. Romand. “The respiratory change in preejection period: a new method to predict fluid responsiveness.” Journal of Applied Physiology 96.1 (2004): 337-342, Nandi et al. showed that PEP is sensitive to respiration, with a lengthening of PEP during inspiration and a shortening during expiration. Nandi, Priya S., Veronica M. Pigott, and David H. Spodick. “Sequential cardiac responses during the respiratory cycle: patterns of change in systolic intervals.” CHEST Journal 63.3 (1973): 380-385. Thus, PEP is a preload-dependent time interval that will lengthen when a fluid responsive subject encounters a preload decrease. As shown by Spodick et al., PEP is largely insensitive to changes in heart rate. Spodick, David H., et al. “Systolic time intervals reconsidered: reevaluation of the preejection period: absence of relation to heart rate.” The American journal of cardiology 53.11 (1984): 1667-1670.

LVET.

The left ventricular ejection time (LVET) defines the duration of ventricular ejection, i.e., from the aortic valve opening (AVO) to the aortic valve closure (AVC). AVO can be determined from the first heart sound as defined above. AVC is defined as the start of the second heart sound (S2).

Alternatively, the LVET can be determined from PPG pulse waveforms recorded at peripheral sites such as the finger or the ear. As shown by Quarry-Pigott et al., and later by Chan et al., careful analysis of the derivative PPG waveform can identify transition points or peaks that correspond to the opening and closing of the aortic valve. Quarry-Pigott, Veronica, Raul Chirife, and David H. Spodick. “Ejection Time by Ear Densitogram and Its Derivative.” Circulation 48.2 (1973): 239-246. Chan, Gregory S H, et al. “Automatic detection of left ventricular ejection time from a finger photoplethysmographic pulse oximetry waveform: comparison with Doppler aortic measurement.” Physiological measurement 28.4 (2007): 439. In one approach, shown in FIG. 4, LVET is defined as the interval between the first and third peaks in the first derivative of the PPG waveform. In a second approach LVET is defined as the interval between the first and third peaks in the third derivative of the PPF waveform. When LVET can be determined from the PPG, PEP can be computed as PEP=EMS−LVET, where EMS defines the time interval from the ECG R wave to the second heart sound.

The LVET is a second STI that allows assessment of ventricular performance. LVET is strongly affected by preload (and hence stroke volume), with larger stroke volumes lengthening LVET. LVET is also affected by heart rate (HR), with faster heart rates reducing LVET. Weissler et al suggest the use of the left ventricular ejection time index (LVETI), which is computed as LVETI=1.6×HR+LVET, where HR is the heart rate in beats/min. Any hemodynamic assessments based on LVET can also be based on the heart rate corrected index, LVETI.

PTT.

In general, given an arterial segment of length D, the PWV is defined as:

$$PWV = \frac{D}{PTT}$$

Where PTT is the Pulse Transit Time, i.e. the time that a pressure pulse will require to travel through the whole segment. The pulse transit time (PTT) indicates the duration required for the pulse wave to propagate through the arterial tree. The PTT begins with the opening of the aortic valve and ejection of blood from the left ventricle, and concludes when the pulse wave foot has reached the peripheral recording site. In practice, PTT is measured between to vascular locations such as the carotid artery and the femoral artery or hand and foot of the patient.

PTT is sensitive to the distance (D) traveled by the pulse wave and to the pulse wave velocity (PWV). For a single individual and PPG recording site, D is constant. In contrast, PWV will be affected by changes in blood pressure. This is due to the dependence of PWV on arterial compliance and the reduction of arterial compliance at higher distending pressures. In simple terms, a higher blood pressure causes the arteries to become more resistant to stretch, and thus increases the travel velocity of the pulse wave. As shown by Gribbin et al., the relationship between blood pressure and PWV is strongly linear within an individual. Gribbin, Brian, Andrew Steptoe, and Peter Sleight. “Pulse wave velocity as a measure of blood pressure change.” Psychophysiology 13.1 (1976): 86-90.

Pulse Amplitude.

Pulse amplitude describes the size of the pulse waveform as detected with the PPG. Pulse amplitude can be computed as pulse height, from the foot of the waveform to the peak, or as area under the curve (AUC), the area under the PPG waveform from foot-to-foot. In our experience, AUC can be a more robust measure of pulse amplitude. Over long time periods, changes in pulse amplitude can reflect many factors including vascular tone, body position, and PPG sensor attachment. However, over short time periods (minutes) where body position and vascular tone are relatively constant, the primary factor affecting pulse amplitude is pulse pressure, which is directly influenced by stroke volume.

Pulse Contour.

The pulse contour describes the shape of the pulse waveform. The peripheral pulse waveform reflects a summation of the primary wave and secondary waves that arise from various reflections in the vascular tree. Changes in volume status and stroke volume impact the size of reflected waves relative to the primary wave. Thus, pulse contour analysis can be used for hemodynamic assessment. Because the pulse waveform varies in amplitude, frequency, and shape quantification methods vary and include frequency analysis, wavelet transformation, various decomposition methods and curve fitting. An example curve fitting approach uses a mixture of Gaussians which capture the relative timing and amplitude of primary and reflected pulse waves. The resulting model parameters can be used to assess volume status.

Subject Perturbations

For the purpose of determining a patient's diabetes status or hypertensive status, the patient may be required to engage on activities that improve the information content for the assessment. In general, the perturbations create changes in transmural pressure and enable the development of compliance curves.

Controlled Breathing.

Embodiments of the current invention use controlled breathing to create repeatable intrathoracic perturbations. The process does not include mechanical ventilation and is distinguished from common spontaneous breathing in that the breathing activity is volitional. Controlled breathing represents a volitional activity of the patient and includes properties of pace (or rate) as well as pressure. The result is a systematic perturbation that changes intrathoracic pressure in a defined and repeatable manner resulting in stroke volume changes.

The controlled breathing system can be configured so that pressures are the same on inhalation and exhalation (symmetric) or different on inhalation and exhalation (asymmetric). Note that the resistance pressure can be modified to facilitate different defined intrathoracic pressure changes. The resistance pressures can be used to magnify normal changes in intrathoracic pressure leading to larger changes in venous return resulting in large changes in stroke volume. These larger than normal physiology changes in venous return subsequently create larger changes in stroke volume and facilitate the determination of central compliance.

Controlled breathing can be implemented at zero resistance or at multiple defined levels. A significant benefit of a controlled breathing protocol at different resistance levels is the creation of a moderately consistent breathing process with multiple levels of evaluation.

In summary, embodiments of the invention can utilize a controlled breathing system that creates defined and repeatable intrathoracic pressure changes by utilizing a breathing device. Vascular compliance parameters can be obtained at multiple pressure settings that cause changes in stroke volume and facilitate a more accurate assessment of the patient's physiological status.

Self-Initiated Positional Changes.

Passive leg raising (PLR) is a test that translocates transferring a volume of approximately 300 mL of venous blood from the lower body toward the right heart. This results in a stroke volume change where the hydrostatic pressure changes in the upper body are minimized.

Changes in stroke volume can be created by having the patient perform self-initiated positional changes. These positional changes cause a decrease or increase in venous return in an acceptably repeatable fashion. For example, a significant decrease in venous return can be achieved by have the patient move from the supine position to the seated position to the standing position. These positional changes will result in stroke volume changes.

Arterial Stiffness

Diabetes Changes Arterial Compliance.

Diabetes mellitus is one of the major cardiovascular risk factors and has been associated with premature atherosclerosis. There are numerous studies showing that patients suffering from Type 1 diabetes and patients suffering from Type 2 diabetes have an increased arterial stiffness compared to controls. The increase in arterial stiffening in patients with Type 1 and Type 2 diabetes mellitus is evident even before clinical micro- and macrovascular complications occur, being already present at the stage of impaired glucose tolerance. The mechanism of increased arterial stiffness relates to changes in elastin and collagen within the walls; the elastin fibers become fractured and collagen deposition is increased. Moreover, elevated glucose levels promote the formation of advanced glycation end-products, which has been associated with changes in the vessel walls.

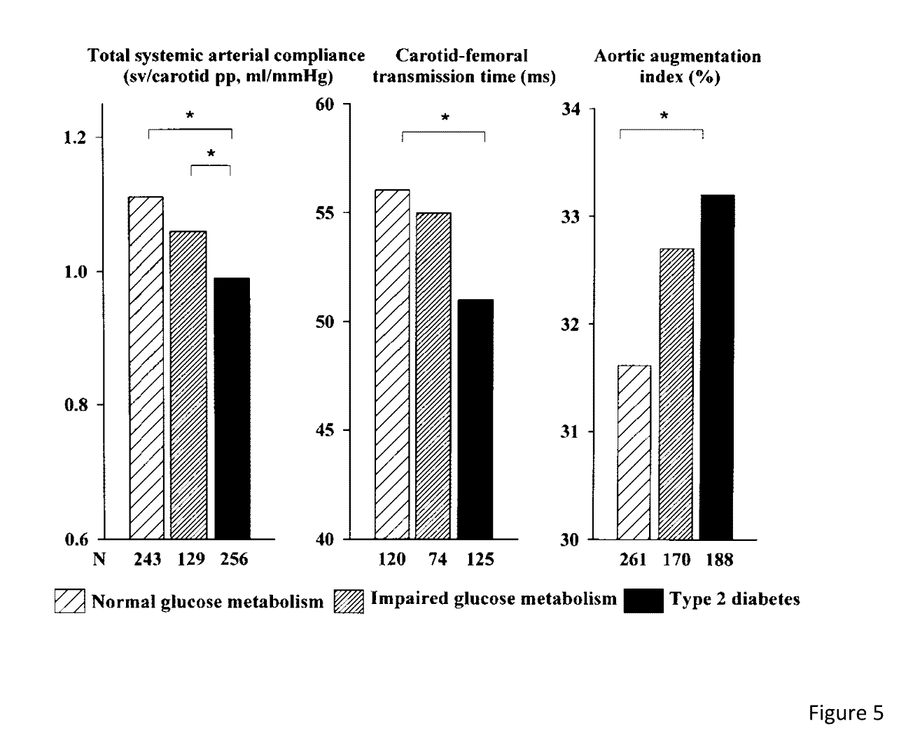

Schram, Miranda T., et al. “Increased central artery stiffness in impaired glucose metabolism and Type 2 diabetes the Hoorn Study.” Hypertension 43.2 (2004): 176-181, and Stehouwer, C. D. A., R. M. A. Henry, and I. Ferreira. “Arterial stiffness in diabetes and the metabolic syndrome: a pathway to cardiovascular disease.” Diabetologia 51.4 (2008): 527-539, have studied and published on the relationship between arterial compliance and the development of diabetes. The authors conducted a population-based study of 619 individuals and assessed central artery stiffness by measuring total systemic arterial compliance, aortic pressure augmentation index, and carotid-femoral transit time. After adjustment for sex, age, heart rate, height, body mass index, and mean arterial pressure, Type 2 diabetes mellitus (DM-2) was associated with decreased total systemic arterial compliance, increased aortic augmentation index, and decreased carotid-femoral transit time. The work of Schram et al. examined the three different measures of compliance in three groups with degrees of glucose control: normal, impaired glucose metabolism and Type 2 diabetes. The results of this analysis are shown in FIG. 5. Examination of FIG. 5 shows a relationship between increasing diabetes severity and decreased arterial compliance. Other researchers have shown that arterial stiffness increases with deteriorating glucose tolerance, (Henry, R. M. a, Kostense, P. J., Spijkerman, a. M. W., Dekker, J. M., Nijpels, G., Heine, R. J., . . . Stehouwer, C. D. a. (2003). Arterial stiffness increases with deteriorating glucose tolerance status: The Hoorn study. Circulation, 107(16), 2089-2095.) Stehouwer et al. provide valuable summary of the relationship between arterial stiffness and metabolic syndrome, (Stehouwer, C. D. a, Henry, R. M. a, & Ferreira, I. (2008). Arterial stiffness in diabetes and the metabolic syndrome: A pathway to cardiovascular disease. Diabetologia, 51(4), 527-539.)

Diabetes has a preferential impact on the central vasculature, as shown by Kimoto et al (Kimoto et al., 2003). The authors state that diabetic patients had greater PWV than the healthy subjects in the four arterial regions (heart-carotid, heart-brachial, heart-femoral, and femoral-ankle segments), and the effect of diabetes on PWV was greater in the heart-carotid and heart-femoral segments (central) than in the heart-brachial and femoral-ankle regions (peripheral). PWV increased with age in the four arterial regions, and the effect of age on PWV was greater in the central than in peripheral arteries. In multiple regression analysis, age and systolic blood pressure had significant impacts on PWV of the four regions, whereas diabetes was significantly associated only with PWV of the central arteries. The current invention provides a system and method for assessment of central compliance.

The following observations can be useful in understanding the present invention. Arterial stiffness is increased in Type 1 diabetes and is an early phenomenon that occurs before the onset of clinically overt micro- or macrovascular complications. Arterial stiffness is increased in Type 2 diabetes and is an early phenomenon that occurs in the impaired glucose metabolism state. The presence of micro- and macrovascular complications is associated with a further increase in arterial stiffness. Arterial stiffness is also increased in the metabolic syndrome and in insulin-resistant states; subtle changes in metabolic variables (not fully developed diabetes) affect arterial stiffness from an early age. Diabetes is a disease of accelerated arterial aging, as shown by stiffer arteries and consequent steeper increases in pulse pressure with age in individuals with pre-diabetes or diabetes.

Despite the strong trends at the population level, as shown in FIG. 5, the ability to use arterial stiffness measures as a screening tool for individuals has not been demonstrated due to large inter-individual variability and inadequate information. For example, in the 2004 study by Schram et al, although the mean carotid-femoral transit time between the normal glucose metabolism group and Type 2 diabetes sample was (statistically) significantly different, the variability of measurements within each group was very large. The mean transit time ±the standard deviation for normal and Type 2 diabetes groups was 56±17 ms and 53±17 ms, respectively. Thus, the individual values from the distributions are highly overlapping. This degree of overlap in pulse wave velocity and parameters associated with arterial stiffness would reduce the specificity with corresponding negative impact on sensitivity. Such a degree of overlap would preclude the use of this test as a diabetes screening test.

A major contributor to this overlap is the influence of blood pressure (BP) on PWV. The theoretical framework that outlines the relationship between PTT and blood pressure is well-known and defined by the Bramwell and Hill equation, which connects PWV with the volume of the vessel and the compliance of the vessel wall at that volume. An acute rise in blood pressure will cause the expansion of the vessel, resulting in a reduction in compliance (increased stiffness). This increased stiffness causes increased PWV. Equivalently, a fall in BP will reduce vascular stiffness and consequently the PWV will become slower. Thus, the same vessel will exhibit different pulse wave velocities when the pressure in the vessel is different.

Pressure Influences Pulse Wave Velocity.

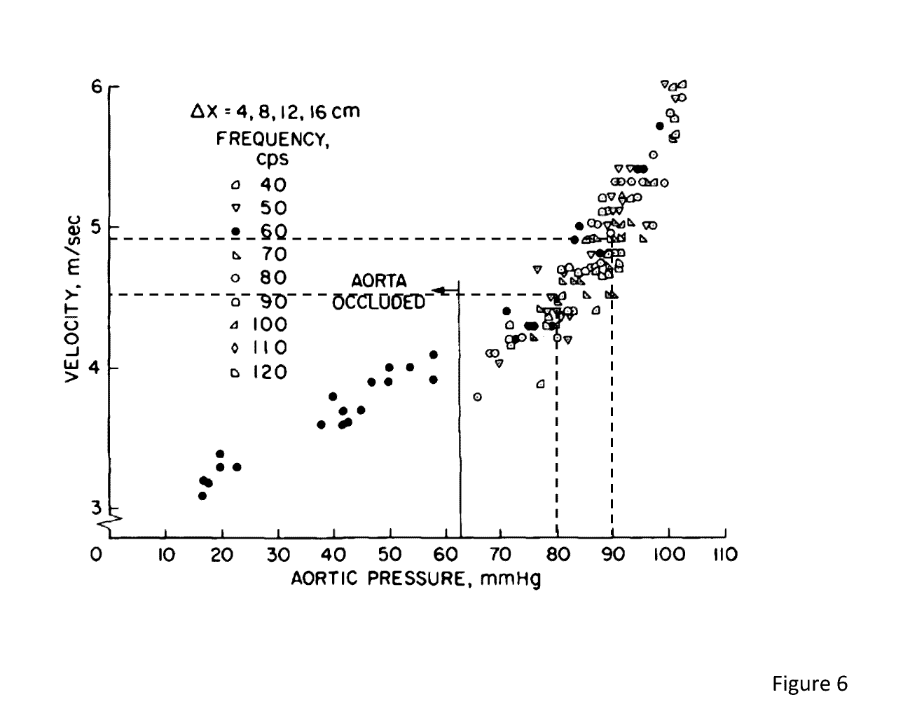

The impact of pressure on pulse wave velocity has been examined in detail by Anliker et al. (Anliker, M., Histand, M. B., & Ogden, E. (1968). Dispersion and attenuation of small artificial pressure waves in the canine aorta. Circulation Research, 23(4), 539-551). Examination of FIG. 6 shows the influence of pressure on pulse wave velocity. In this experiment, a change of 10 mmHg results in a roughly 0.4 m/sec change in the velocity (dashed lines added to figure to emphasize the influence).

The inability to use PWV as an effective measure of true arterial compliance has been recognized and efforts have been made to develop systems that are pressure insensitive. Shirai et al. have developed the cardio-ankle vascular index (CAVI) as a methodology that minimizes pressure dependency (Shirai, K., Utino, J., Otsuka, K., & Takata, M. (2006). A novel blood pressure-independent arterial wall stiffness parameter; cardio-ankle vascular index (CAVI). Journal of Atherosclerosis and Thrombosis, 13(2), 101-107). As stated by the authors, the problem with PWV measurements in clinical use is the velocity dependence on blood pressure. The authors address this problem by using two blood pressure cuffs located at the brachial artery and the tibial artery. Using a mathematical formula initially derived from the Bramwell-Hill formula, the method has a goal of reduced pressure sensitivity. The result is a cardio-ankle vascular index that reflects the stiffness of the aorta, femoral artery and tibial artery and is reported to be independent of blood pressure. In summary, Shirai et al have defined a methodology based upon the use of multiple blood pressure cuffs that creates a singular assessment of arterial stiffness with reduced sensitivity to blood pressure.

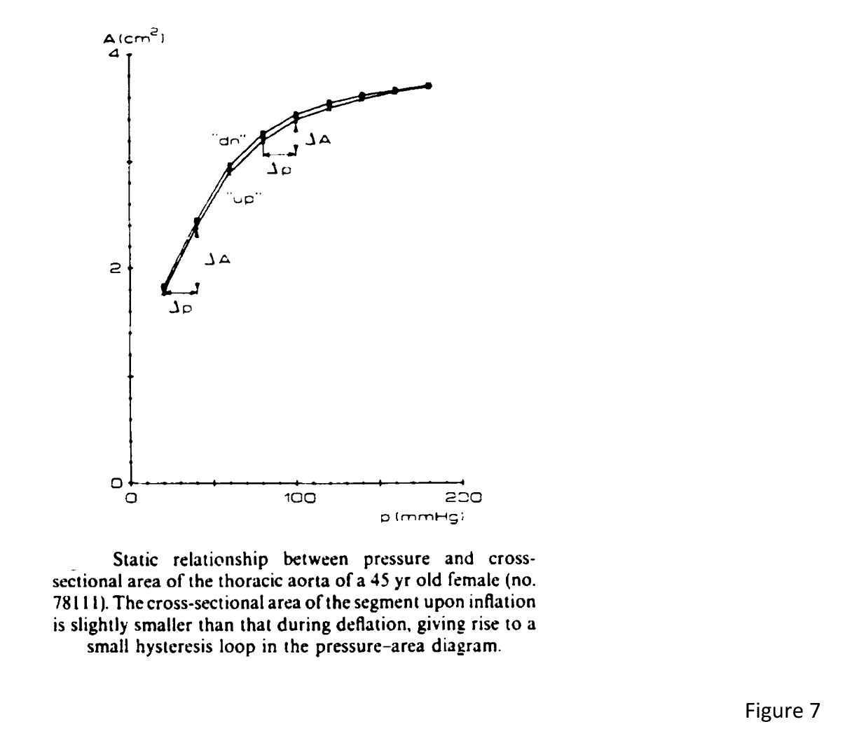

The actual characterization of arterial stiffness by a singular metric is problematic. The seminal work in arterial stiffness characterization was conducted by Langewouters. The work involved a careful examination of excised thoracic and abdominal aortas over age ranges between 30 and 88 years. The use of excised aortas enabled pressure normalization, so true compliance curves could be created. The work created standardized compliance curves and resulted in the development of the arctangent compliance model (Langewouters, G. J., Wesseling, K. H., & Goedhard, W. J. (1984). The static elastic properties of 45 human thoracic and 20 abdominal aortas in vitro and the parameters of a new model. Journal of Biomechanics, 17(6), 425-435). The arctangent model describes the nonlinear relationship between pressure and vessel area. From a physiological perspective as pressure increases, the diameter of the vessel cannot continue to increase otherwise rupture would occur. Therefore, the vessel becomes increasingly stiff as the diameter (or volume) increases. FIG. 7 is a representative example of the nonlinear relationship between pressure and cross-sectional area of the arterial vessel, reproduced from Langewouters' seminal study.

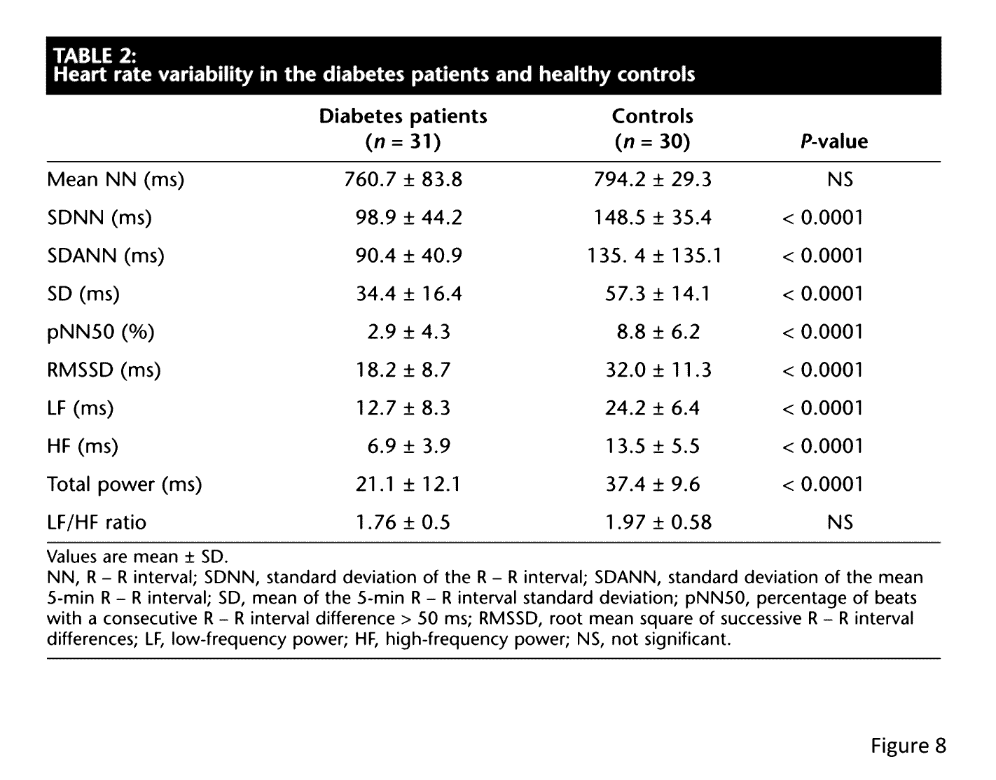

Limitations of using a singular parameter for arterial stiffness were emphasized by Tardy et al. As stated in the abstract, “the non-linear elastic response of arteries implies that their mechanical properties strongly depend on blood pressure. Thus, dynamic measurements of both the diameter and pressure occurs over the whole cardiac cycle are necessary to characterize properly the elastic behavior of an artery”, (Tardy, Y., Meister, J. J., Perret, F., Brunner, H. R., & Arditi, M. (1991). Non-invasive estimate of the mechanical properties of peripheral arteries from ultrasonic and photoplethysmographic measurements. Clinical Physics and Physiological Measurement: An Official Journal of the Hospital Physicists' Association, Deutsche Gesellschaft Fur Medizinische Physik and the European Federation of Organisations for Medical Physics, 12(1), 39-54.). To demonstrate this point, the authors utilized ultrasound for determination of the internal diameter of the peripheral artery and a continuous finger blood pressure measurement system. The resulting pressure and diameter information was used to create diameter-pressure curve relationships that were fit using the arctangent method of Langewouters. Tardy demonstrates the limitation of a singular or mean compliance measurement on page 50 by emphasizing the necessity of obtaining compliance curves in order to compare different vessels meaningfully. The authors provide a specific example of arterial compliance measured in two subjects. “One method was based only on extreme values of pressure and cross-section during systole and diastole (mean compliance). The other method relied on our continuous compliance curve approach. Using extreme values only, the compliance values for these two subjects appear similar (i.e. 0.156 mm² kPa⁻¹ or 0.022 mm² mmHG⁻¹), but once their compliance-pressure curves are established it appears that the dynamic behavior of these vessels is different”, see FIG. 8 of the publication. The need to utilize an arterial compliance function for effective arterial compliance characterization was also recognized by Hasson et al. (1984) and Megerman et al (1986).

The standard use of arterial compliance or arterial stiffness refers to a general characteristic of the vessel without regard for the conditions of the measurement, specifically the blood pressure or transmural pressure at the time of the measurement. Many authors simply refer to arterial compliance as a point assessment without regard for measurement conditions. As shown by Langewouters and others, the characterization of arterial compliance is in fact a function that relates pressure to volume. To address the inaccuracy of using a single point compliance measurement, the term “compliance-state” will be used herein to define compliance at a defined pressure. The use of “compliance-state” addresses a major limitation of prior screening work by defining the compliance of the vessel under a defined set of conditions.

The new method of screening for diabetes determines both a central compliance curve and a peripheral compliance curve by changing transmural pressure in a measurable manner. Central compliance is determined by using changes in thoracic pressure to create stroke volume changes that result in transmural pressure changes. Peripheral compliance is determined by using hydrostatic pressure changes to change the transmural pressure.

The resulting method requires no patient adherence with fasting requirements, does not require a blood draw, provides immediate results, and is based upon physiological parameters that are leading indicators for the development or presence of diabetes.

Example embodiments of the invention incorporate multiple inventive steps. It is recognized that each improvement can be used independently or in conjunction with other improvements to create a diabetes assessment and hypertension assessment system that is a dramatic improvement over conventional approaches in terms of cost, convenience and performance.

In summary, historical publications have independently demonstrated two significant problems associated with a singular or point compliance assessments based upon pulse wave velocity. First, historical measurements do not account for variances in blood pressure which are known to influence PWV. Second, the use of a singular arterial compliance measurement is inadequate, as it characterizes only the instantaneous “compliance-state” of the artery. The present invention addresses both deficiencies in an elegant and easy to implement manner.

Arterial Stiffness Changes

The arterial wall stiffness depends on the structural elements within the arterial wall, for example muscle, elastin and collagen. In addition to diabetes, there are several other conditions that can contribute to increasing vascular stiffness. The stability, resilience, and compliance of the vascular wall are dependent on the relative contribution of its prominent scaffolding proteins: collagen and elastin. The relative content of these molecules is normally held stable by a slow, but dynamic, process of production and degradation. Dysregulation of this balance, mainly by stimulation of an inflammatory milieu, leads to overproduction of abnormal collagen and diminished quantities of normal elastin, which contribute to vascular stiffness. Increased luminal pressure, or hypertension, also stimulates excessive collagen production. In addition to diabetes, chronic renal disease is known to cause vascular stiffening. The influences of age and hypertension are discussed separately below.

Arterial Stiffness is Influenced by Age

Increasing age leads to increase arterial stiffness as shown by Millasseau et al. “Determination of Age-Related Increases in Large Artery Stiffness by Digital Pulse Contour Analysis.” Clinical Science (London, England: 1979) 103, no. 4 (2002): 371-77. Stiffness changes associated with aging are due to the fatiguing effects of cyclic stress acting over many decades on the inherent nonliving elastic fibers and resulting in their fracture and separation.

Arterial Stiffness is Influenced by Hypertension.

Hypertension is known to accelerate arterial stiffness. With hypertension, the change in arterial stiffness is strongly influenced by transmural distending pressure and by mean blood pressure. The increase in pressure is associated with increased vascular resistance and associated structural changes. In general terms, stiffness changes due to diabetes are associated with advanced glycation end products, which lead to a cross-linking of the collagen and a general change in the elastic nature of the collagen. These changes affect the central arteries more specifically than the peripheral arteries. Hypertension results in an accelerated stress fatigue. From a clinical measurement perspective, hypertension affects both the central vasculature as well as the peripheral vasculature whereas diabetes has a greater influence on the central vasculature.

Autonomic Function

Autonomic Function Changes in the Presence of Diabetes and Hypertension.

Diabetes is one of the main causes of autonomic neuropathy. Cardiovascular autonomic neuropathy can cause abnormalities in the control of heart rate, with loss of its variability, decreased baroreceptors sensitivity, and late changes in vascular dynamics. In healthy individuals, heart rate has a high inter-beat interval variability which fluctuates with breathing. Generally, heart rate increases during inspiration and decreases during expiration. Diabetes reduces heart rate variability (Kudat, H., Akkaya, V., Sozen, a B., Salman, S., Demirel, S., Ozcan, M., . . . Guven, O. (2006). Heart rate variability in diabetes patients. The Journal of International Medical Research, 34(3), 291-296.) Examination of Table 2 (see FIG. 8) of the prior reference shows strong population differences but overlapping individual measurements between diabetics and controls.

Cardiovascular autonomic dysfunction is also associated with essential hypertension and is associated with parasympathetic over-activity. Multiple studies have reported decreased heart rate variability among individuals with hypertension. The Atherosclerosis Risk in Communities (ARIC) study examined this relationship over a nine-year period, (Schroeder, E. B., Liao, D., Chambless, L. E., Prineas, R. J., Evans, G. W., & Heiss, G. (2003). Hypertension, Blood Pressure, and Heart Rate Variability: The Atherosclerosis Risk in Communities (ARIC) Study. Hypertension, 42(6), 1106-1111). The evaluation of autonomic nervous system function involves measures of heart rate variation at rest and in response to deep respiration, Valsalva maneuver, position changes and apneic facial immersion. The parameters used to quantify heart rate variability are well documented via a task force on this topic (Guidelines. (1996). Guidelines Heart rate variability. European Heart Journal, 17, 354-381). This document is incorporated by reference since the document provides metrics for parameterizing heart rate variability. These metrics as well as other metrics that quantify heart rate variability can be used to effectively define various characteristics of autonomic function as well as the presence of autonomic neuropathy.

Although both diabetes and hypertension result in decreased heart rate variability, there are differences in the pathophysiology associated with disease detection. Small differences in the parameters defining heart rate variability have been identified between diabetes and hypertension. Istenes et al. conducted research on heart rate variability differences between normal individuals, those with diabetes, those with hypertension and those with both hypertension and diabetes. The results of this analysis showed that multiple parameters were influenced negatively by diabetes whereas hypertension had a negative effect only on low-frequency components. (Istenes. (2010). Quality assessment and improvement in diabetes care—an issue now and for the future. Diabetes/metabolism Research and Reviews, 26(6), 446-447.).

Entropy and Tone Calculation.

One of the complications of diabetes is peripheral neuropathy. Peripheral neuropathy is often diagnosed by measurement of nerve conduction velocity. Karino et al. have demonstrated strong agreement between tone and entropy and sural nerve conduction velocity, (Karino K, Nabika T, Nishiki M, lijima K, Nagai A, Masuda J. Evaluation of diabetic neuropathy using the tone-entropy analysis, a non-invasive method to estimate the autonomic nervous function. Biomed Res. 2009; 30(1):1-6.).

Paced Breathing and Heart Rate Variability.

Heart rate variability is influenced by many aspects of respiratory function. In the article by Tripathi, the influences of respiratory rate, tidal volume, end tidal partial pressure, the time ratio of expiration/inspiration as well as respiratory dead space are all shown to have influence on heart rate variability, (Tripathi, K. (2004). Respiration and heart rate variability: A review with special reference to its application in aerospace medicine. Indian Journal of Aerospace Medicine, 48(1), 64-75.). When testing for autonomic function, it is desirable to obtain a reliable and repeatable test. Heart Rate Variation is influenced by the respiratory cycle due to the mechanics of breathing as well as the autonomic (sympathetic and parasympathetic) nervous system. Paced breathing is often used to create a normalized breathing between patients. Kobayashi et al. investigated this question directly and found that paced breathing can provide some improvement in the reproducibility of heart rate variation measurements although paced breathing may not be necessary depending upon the application. (Kobayashi, Hiromitsu. “Does Paced Breathing Improve the Reproducibility of Heart Rate Variability Measurements?” Journal of Physiological Anthropology 28, no. 5 (2009): 225-30.)

Additional assessments of autonomic function have been conducted by examining the correlation between right and left pulse waveform fluctuations. In the work by Buchs, the PPG signal was measured simultaneously in the fingers and toes of diabetic and nondiabetic individuals. The authors concluded that right-left correlation coefficients of the PPG fluctuations provides a simple and convenient means for assessing the adequacy of sympathetic nervous system function, (Buchs, A., Slovik, Y., Rapoport, M., Rosenfeld, C., Khanokh, B., & Nitzan, M. (2005). Right-left correlation of the sympathetically induced fluctuations of photoplethysmographic signal in diabetic and non-diabetic subjects. Medical and Biological Engineering and Computing, 43(2), 252-257).

Although many changes in physiology have been observed due to diabetes, none of the prior methods has been used to effectively screen for diabetes or pre-diabetes in a previously undiagnosed population. The present invention solves problems due to error sources associated with these measurements and provides a system for diabetes assessment on a previously undiagnosed population.

Compliance Assessment Methods

The diabetes and hypertension assessment system is composed of a simple noninvasive PPG-based technique for measuring in vivo the arterial distensibility over a range of pressures. Changes in arterial pressure are generated via changes in hydrostatic pressure or stroke volume during simultaneous measurement of pulse transit times. Pulse transit times are converted into pulse wave velocities, which have a direct association with arterial distensibility. The determination of pulse wave velocity over a range of transmural pressures creates an arterial compliance curve that can be used to determine the likelihood of diabetes or hypertension.

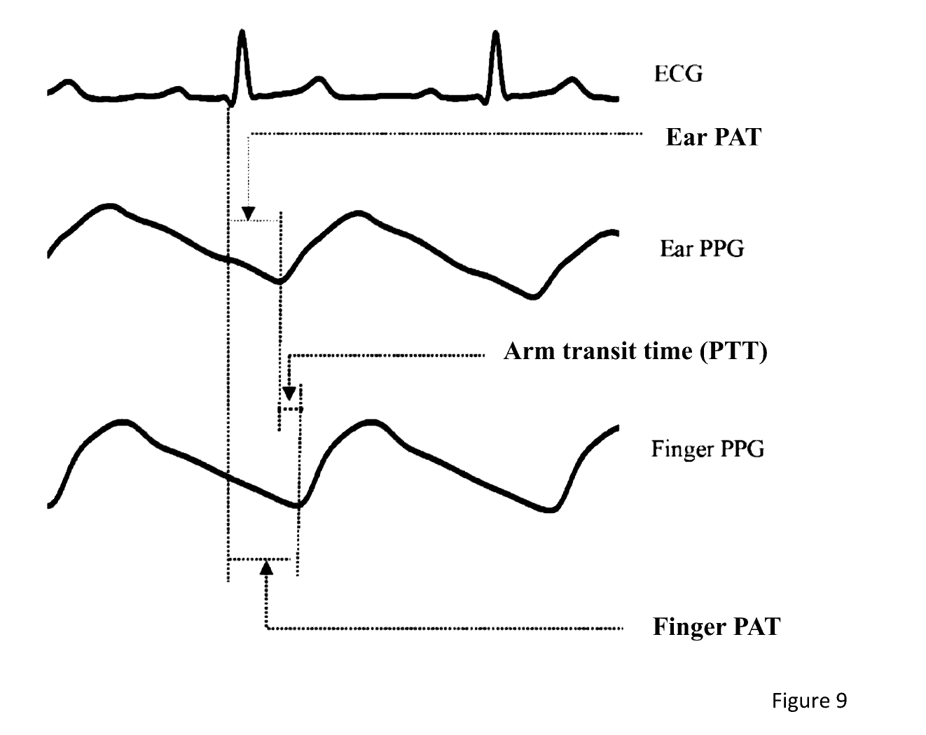

A measurement specific for compliance can be obtained by acquiring two pressure waveforms concurrently at different distances from the heart. The calculation of arm or limb transit times can be done with two PPG measurement devices. FIG. 9 is a graphical illustration of these principles. Examination of the figure also shows the calculation of arm pulse travel time. To minimize the effect of the pre-ejection time, which is common to the simultaneous ear PAT and finger PAT, the ear PATs were subtracted from the finger PATs to obtain the propagation times along the major section of the arm, and is referred to as arm pulse transit time (PTT).

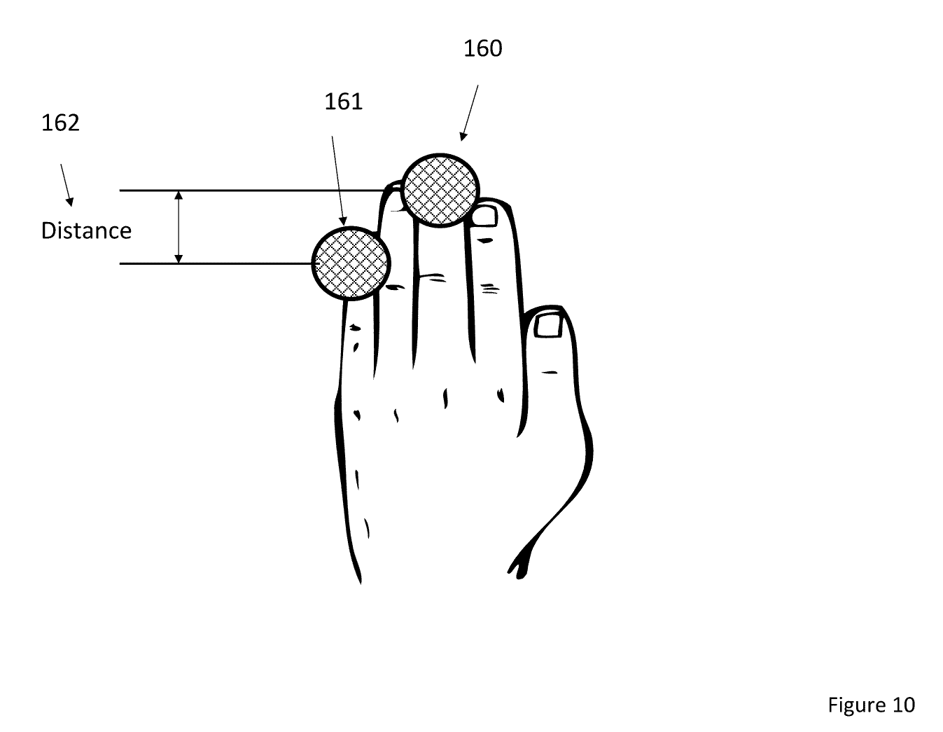

Vascular compliance measurements can include other body locations to include the hand and foot of the subject. More localized compliance measurements can be made by using a wrist to finger measurement, or an index to pinky finger measurement. The determination of a peripheral pulse wave velocity measurement can be used in conjunction or independently with a central pulse wave velocity for diabetes assessment. The combined information can provide insight in cardiac risk, hypertension, disease progression, and effectiveness of treatment. Error! Reference source not found. FIG. 10 shows a configuration of peripheral compliance assessment. Site 160 is a PPG measurement sight that is more distal than the site 161, creating distance difference 162. Standard PWV determinations can be applied on the resulting data.

Determination of Peripheral Arterial Compliance Curve

For a true determination of an individual's cardiovascular condition and diabetes state, it can be desirable to derive a measurement more specific for peripheral compliance. The upper and lower limbs of the subject represent a physical location that can be tested for the determination of peripheral compliance. The determination of peripheral compliance is desired as the pathophysiology of changes due to diabetes in elastic arteries (a.k.a. central arteries) is different than peripheral or muscular arteries. A peripheral compliance curve is a compliance assessment that has preferential specificity for vascular elements that are not the thoracic or abdominal aorta.

With a goal of creating a peripheral compliance curve, the following physiological associations can be leveraged. Changes in arm elevation can be used to create changes in hydrostatic pressure which result in transmural pressure changes. These hydrostatic pressure changes can be used to create a repeatable test scenario with concurrent determination of pulse wave velocity.

The process of measuring pulse wave velocity at different arterial pressures provides information such that a compliance function can be calculated. The process involves recording a PPG signal from a distal site (e.g., the finger) with the recording of an electrocardiogram or a secondary PPG that is located more centrally. In situations where the stroke volume is constant or varies minimally, pulse arrival time can be used as a measure of pulse wave velocity. In these situations, an ECG signal will be combined with a PPG signal from the finger.

As it relates to peripheral compliance, hydrodynamic pressure changes can be created by simply raising the arm relative to the heart. Hydrostatic pressure occurs in the vascular system because of the weight of the blood in the vessels. The effect of gravity, i.e. the positional hydrostatic factor, is equal to p*h*g (dynes/cm²)

where p is the blood density (1.05 g/cm³), g is the acceleration due to gravity (980 cm/sec²) and h is the distance (height) from the reference point in cm. This is negative for levels above the reference point. To convert dynes/cm² into mm Hg the result must be divided by 1360. The pressure is thus decreased in any vessel located above central venous pressure and increased in any vessel below central venous pressure.

Transmural pressure, the difference between internal arterial pressure and external pressure, can be achieved via other means than an arm raise. Another effective non-invasive method for altering local vascular transmural pressure with minimal effect on the remainder of the systemic circulation is to apply pressure external to a limb. Alterations in peripheral transmural pressure can be done through a pressure cuff, placement in the arm in a water bath, placement of the arm in a pressure box, or other external pressure methodologies.

The peripheral compliance curve or function can be generated by the measurement of multiple “compliance-state” measurements. The method uses pulse wave velocity assessment as the mechanism for accessing stiffness. The methodology exploits the effect of several arm positions to characterize compliance as a function of pressure and create a compliance function. The resulting information can be used to calculate the coefficients associated with the physiological exponential elastic model proposed by Hardy and Collins as well as the Langewouters' arctangent model.

Demonstration of Peripheral Compliance Curve

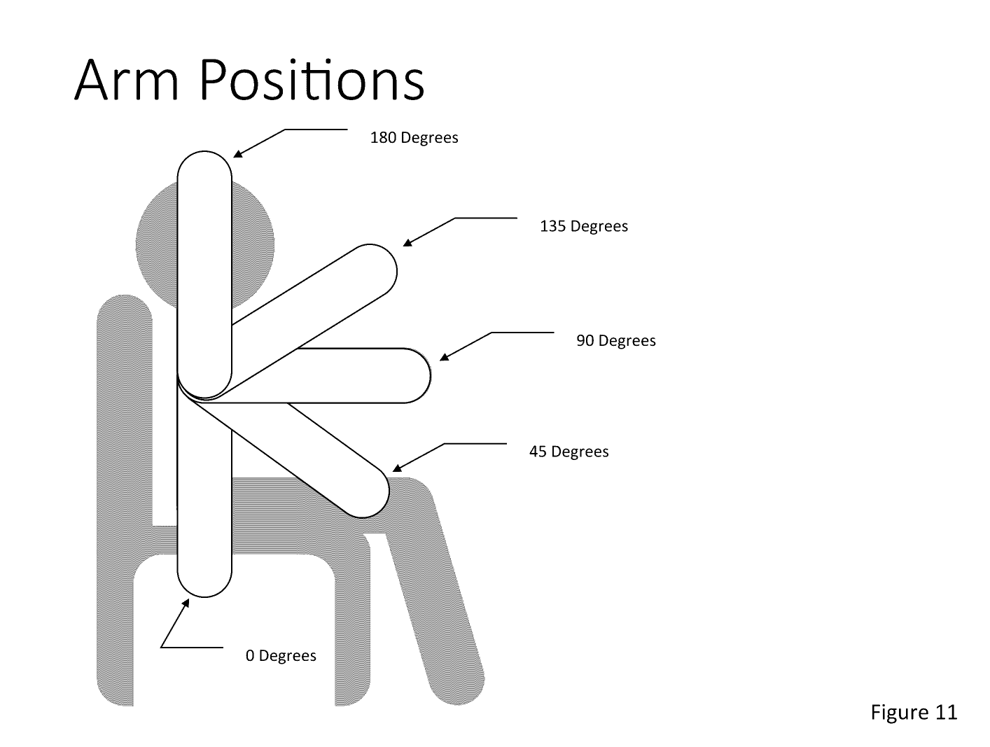

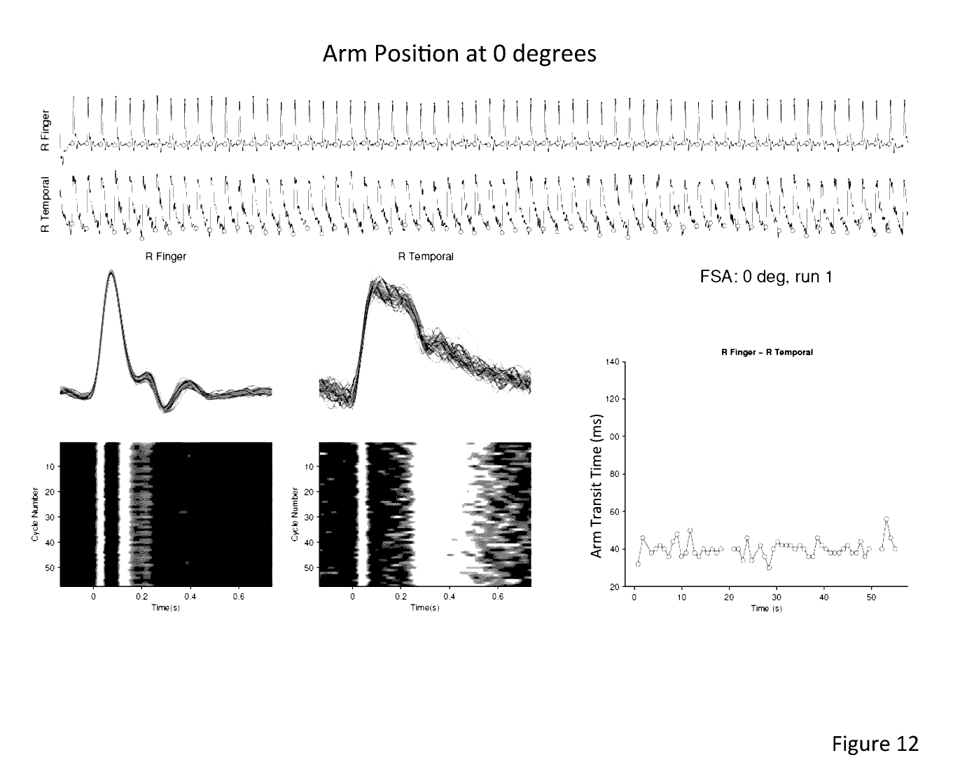

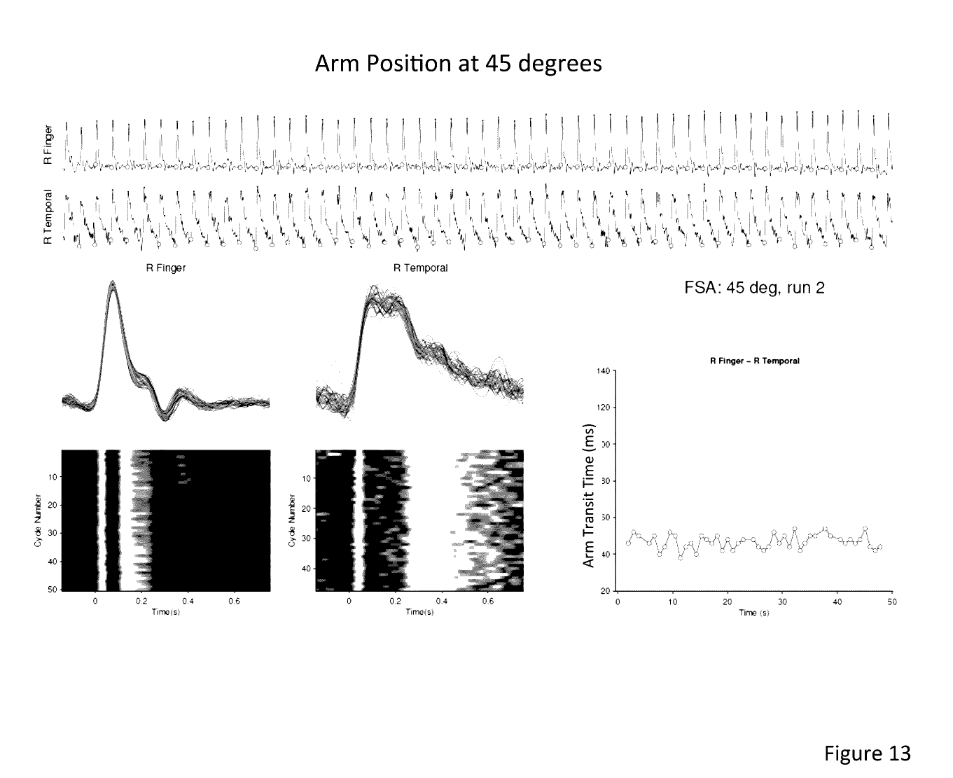

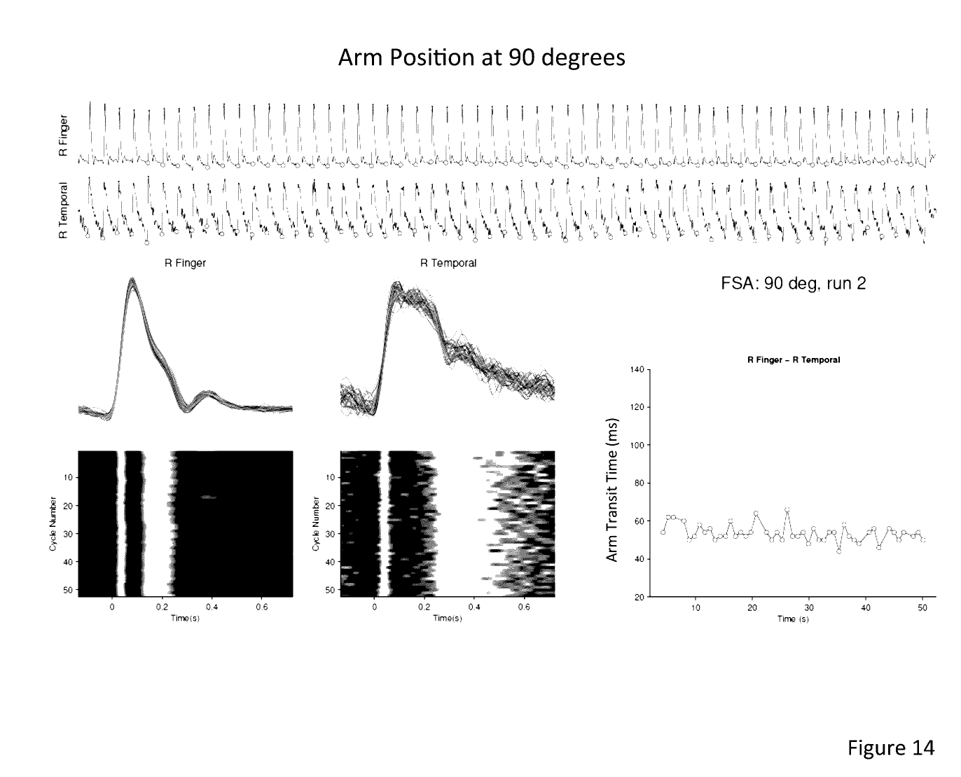

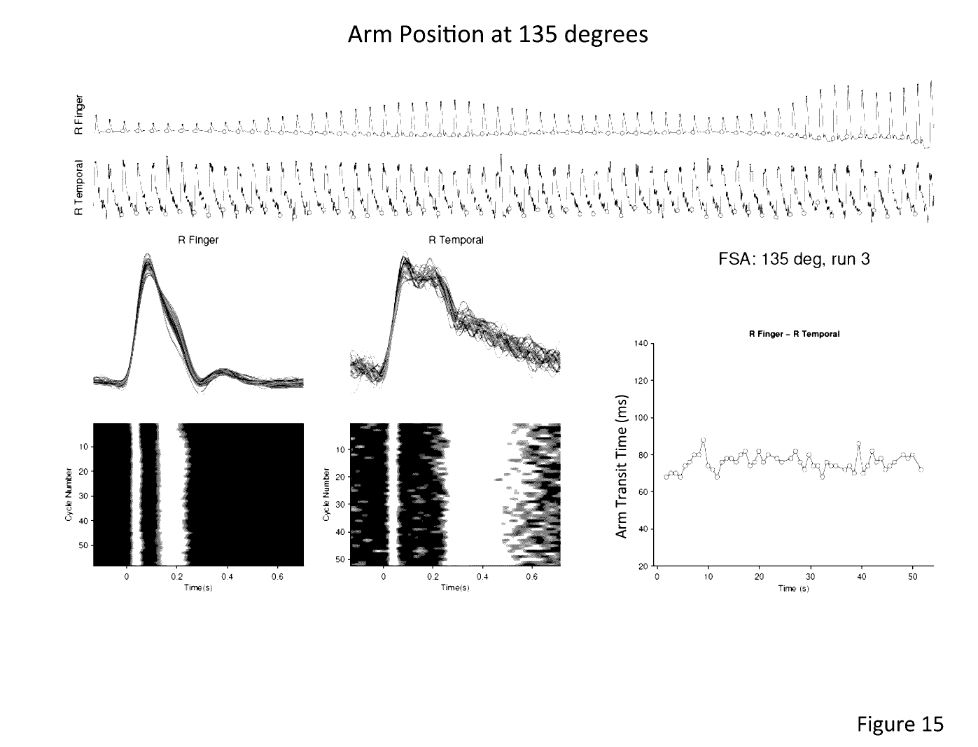

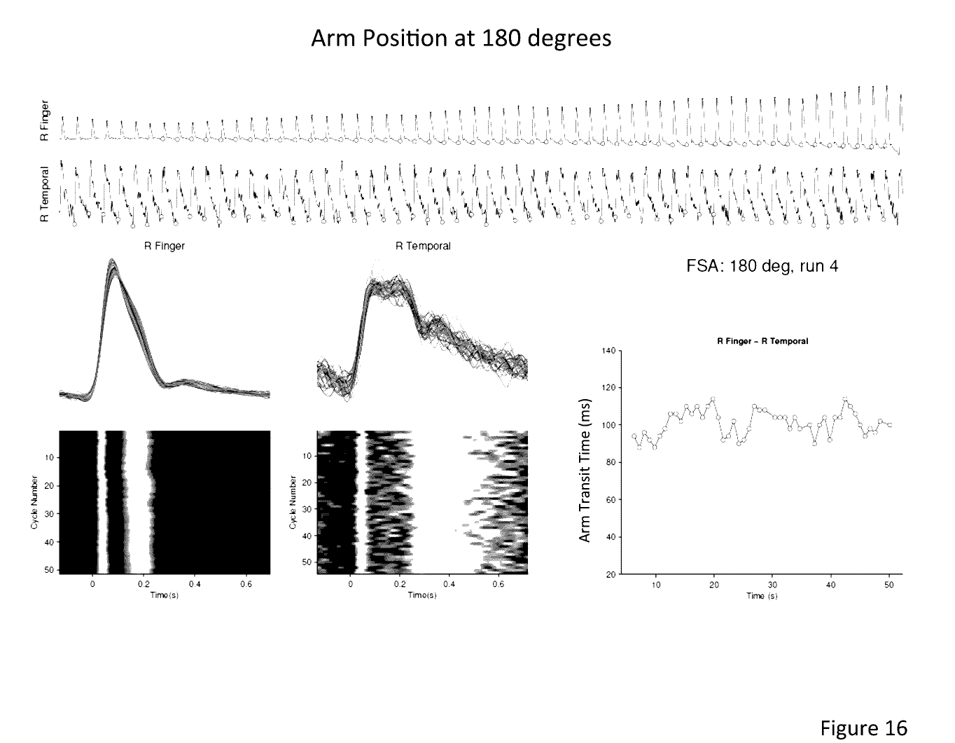

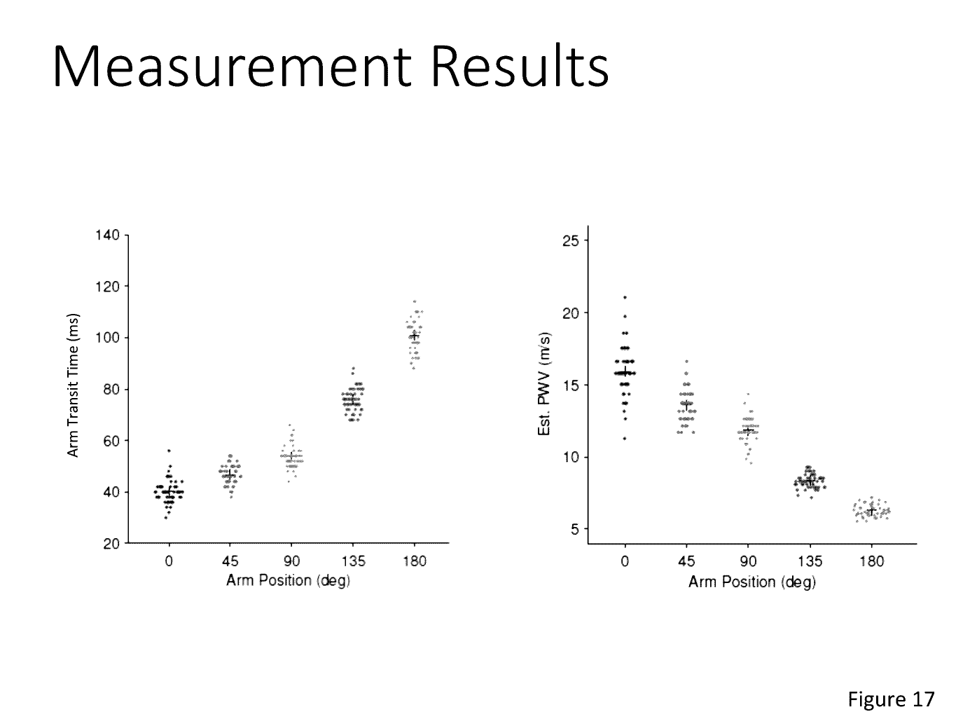

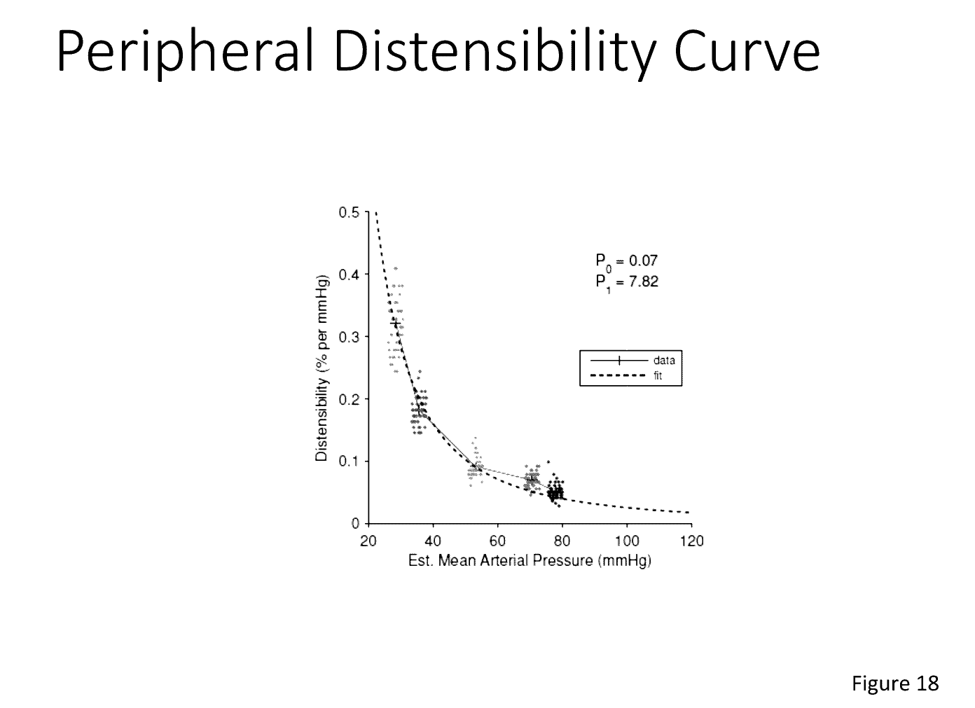

A demonstration of the method was achieved by placing a subject in a supine position with PPG sensors attached to the subject's forehead and finger. A conventional blood pressure measurement was obtained and recorded. Continuous PPG measurements were made for approximately 1 minute with the arm in a 0° position, followed by 45°, followed by 90°, followed by 135° and finally 180°, as shown in FIG. 11. The arm transit time was calculated for each position. FIG. 12, FIG. 13, FIG. 14, FIG. 15, and FIG. 16 show an example of the information obtained as a result of arm location. The figure shows the derivative of the PPG signal from the right finger, the derivative of the PPG signal from the right temporal area over the entire measurement period, top plots. An overlay plot of the derivative waveforms is shown in the center left and a heat map showing the amplitudes over time is provided. The right-hand graph shows the calculated arm transit time over the duration of the measurement, approximately 40 seconds. FIG. 17 shows the arm transit time at each arm position as well as the estimated pulse wave velocity at each position. These plots demonstrate the remarkable sensitivity of pulse transit time as well as pulse wave velocity to changes in arterial pressure. FIG. 18 shows the resulting fit of the data utilizing Langewouters' arctangent model. Several additional points associated with this figure set should be made. A comparison between the right finger pulse waveform at each location shows a dramatic change in all overall pulse shape while the temporal signal remains remarkably constant. The consistency of the temporal wave demonstrates that the movement of the arm does not have significant impact on central compartment pressures. The observed changes in the pulse contour as a function of arm location can be utilized for additional characterization beyond pulse transit time. In summary, the methodology above demonstrates the ability to use multiple “compliance-state” measurements at different pressures as well as create a peripheral compliance function.

Variance arm movement protocols can be used for the generation of a compliance curve. Variances can include a slow continuous motion, up and down tests, tests that have the arm at only two positions, test that require equilibration of the signal, and other arm motion protocols that create frequency modulated changes.

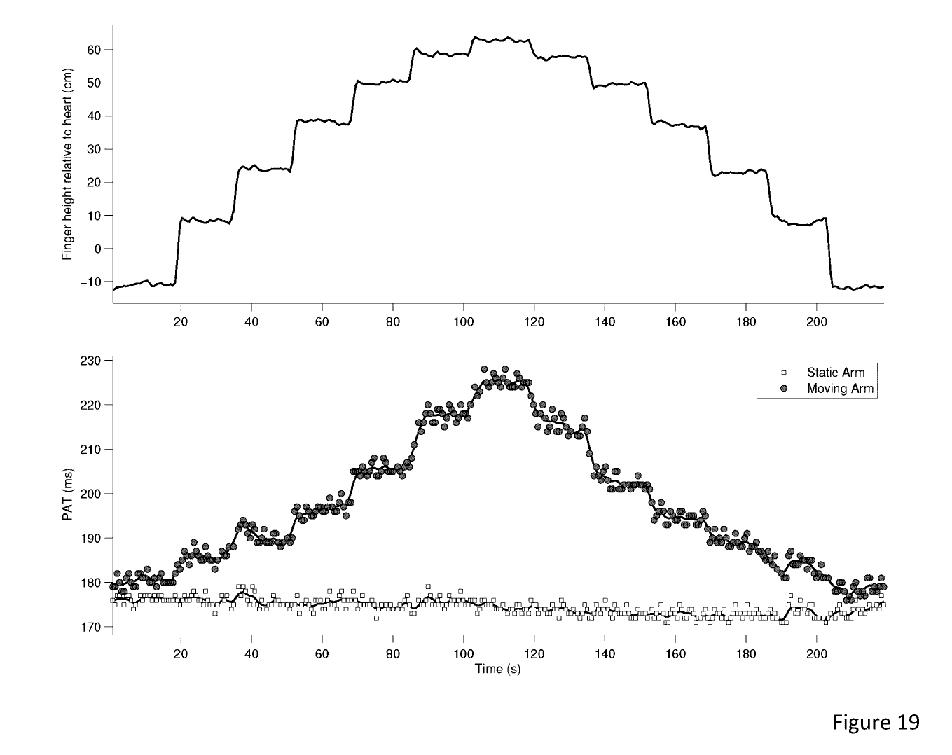

During arm swing testing, the stroke volume from the heart remains quite constant. This characteristic of the test enables the use of pulse arrival time (PAT) as a metric for pulse wave velocity. The PEP period is moderately stable so changes in PAT are almost exclusively due to pulse wave velocity changes associated with transmural pressure changes. FIG. 19 shows the results of an arm swing test with both arms measured. As shown in the figure, the PAT in the static arm is very constant over the test while the PAT in the arm being moved changes as a function of transmural pressure.

Additional Peripheral Compliance Considerations

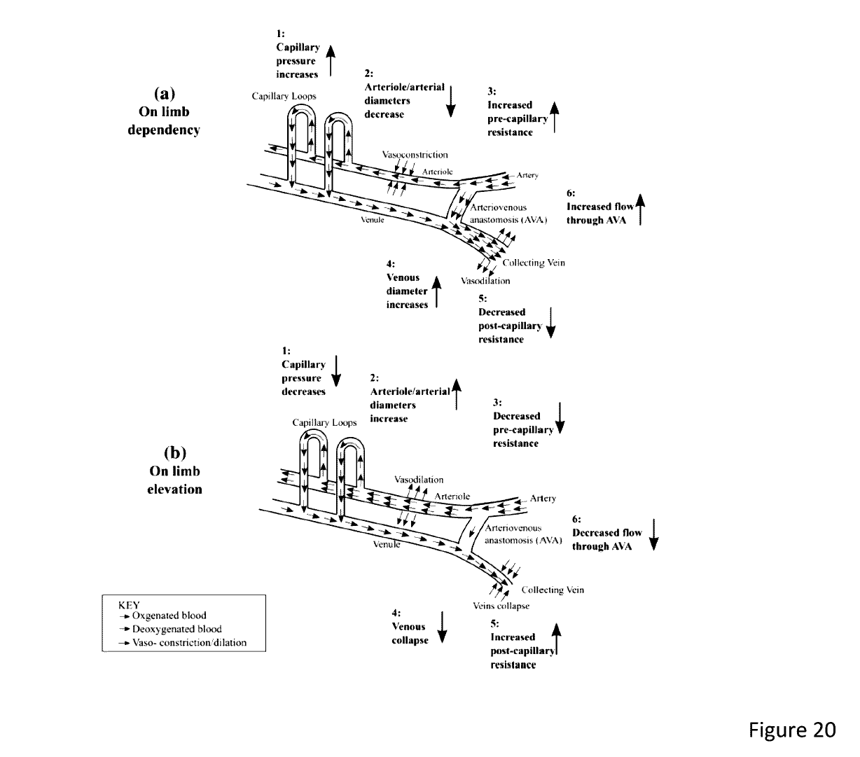

The use of the arm swing or arm elevation method is to create a change in hydrostatic pressure while minimizing other physiological noise sources. The arm vasculature does not act like a static manometer, but instead has blood flow from the heart in all arm positions. Additionally, the system is not composed of rigid vessels and the autonomic system is actively involved in regulating flow through the arm. The vascular changes in the terminal capillary bed of the finger as well as autonomic changes have been characterized by Hickey et al. Hickey, M., Phillips, J. P., & Kyriacou, P. A. (2015). Investigation of peripheral photoplethysmographic morphology changes induced during a hand-elevation study. Journal of Clinical Monitoring and Computing. When the arm is down, capillary pressure is controlled by vasoconstriction resulting in increased pre-capillary resistance. The veins, however are extended due to increased hydrostatic pressure. Additionally, in the end of the finger, there are numerous arteriovenous anastomoses that facilitate general blood flow through the arm and are directly involved in thermoregulation. FIG. 20 reproduced from Hickey et al., illustrates these changes in physiology. With the arm in the down position, vasoconstriction at the precapillary arterioles occurs to effectively reroute blood into the venous system through the arteriovenous anastomoses. In summary, when the arm is below the heart and experiencing higher hydrostatic pressure, capillary flow is restricted, arteriovenous anastomoses flow is high and the veins are dilated. If a photoplethysmogram (PPG) is used to make optical measurements of the tissue, the AC (pulsatile) component of the signal will be small due to smaller arterial pulsations, while the DC (mean) absorbance of the signal will be increased due to the overall increase in blood volume in the tissue. As the arm is elevated, the autonomic nervous system seeks to maintain capillary flow and vasodilation occurs at the precapillary level. Flow through the arteriovenous anastomoses decreases. This physiological change occurs as the veins begin to collapse due to atmospheric pressure being greater than venous pressure resulting in a transmural pressure of zero. This collapse increases the systemic vascular resistance by decreasing the post capillary resistance. Thus, when the arm is above the heart and experiencing reduced hydrostatic pressure, the AC component of the optical PPG signal will be larger while the DC absorbance component of the PPG signal will be decreased.

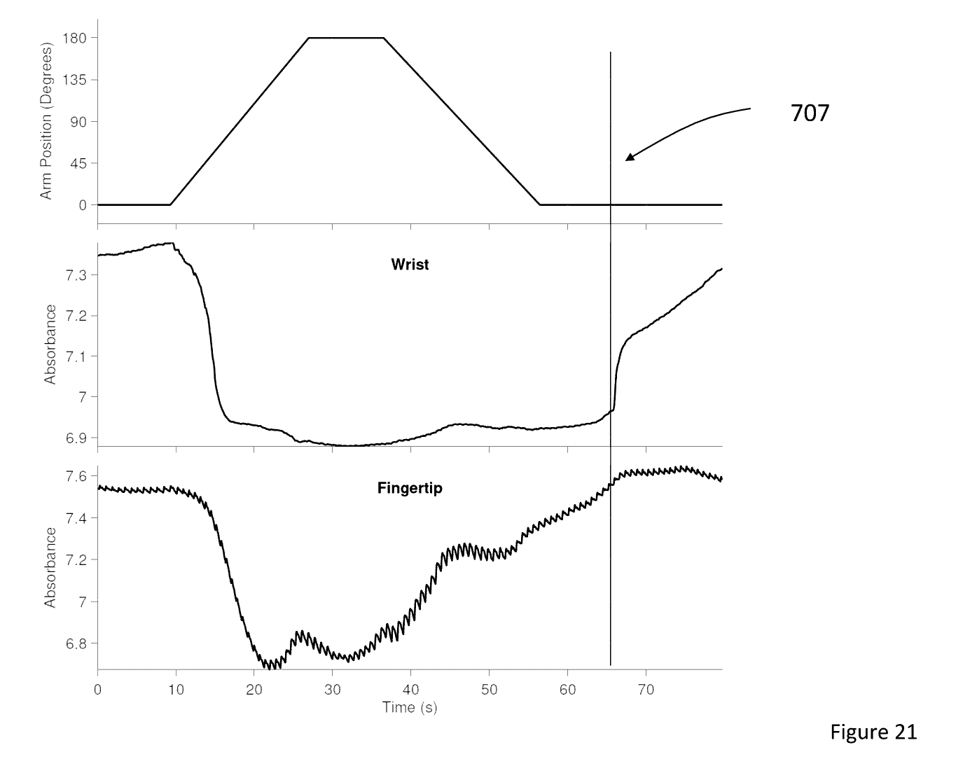

The impact of the above physiological variance can be reduced if desired by sampling an area of the body that is not the terminal capillary bed of a digit and mitigating the influences of venous blood in the optical measurement. FIG. 21 shows the complexity of using the terminal capillary of the finger as a sensor location. Optical absorbance signals at the terminal finger and at the wrist are shown as the arm is rotated from 0 degrees (straight down) to 180 degrees (up). The wrist shows a more constant pulse amplitude while the fingertip shows large variances. Both traces show decreasing absorbance with arm elevation. However, with movement of the arm downward, the venous system requires more time to fill. Thus, the finger DC level increases due to increasing venous blood almost immediately, while the wrist location remains largely uninfluenced. This asymmetric response in DC changes in the wrist can be leveraged to create a less variable optical signal. Specifically, the changes in hydrostatic pressure can be measured on a downward arm movement. Changes in pulse size and the amount of venous blood resident in the optical measurement can be reduced by measuring locations other than the terminal capillary bed of the finger and measuring compliance information during the downward movement of the arm.

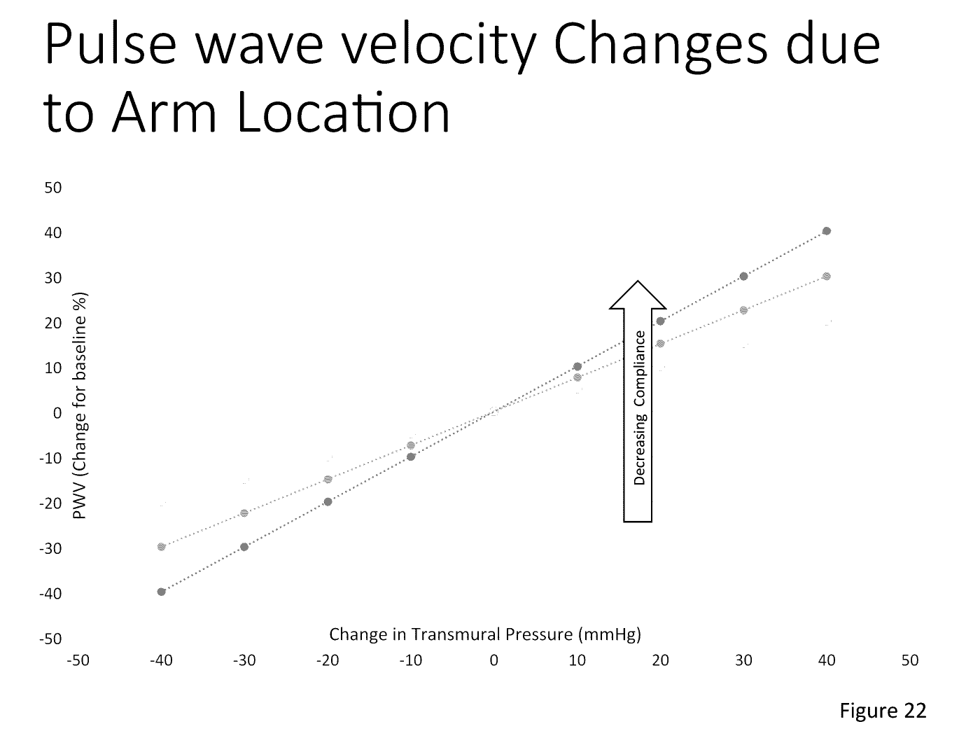

The previously described testing method based upon transmural changes due to arm movement can be conducted without a standard blood pressure determination. Changes in pulse wave velocity can be assessed relative to a defined reference point of datum. FIG. 22 shows an example of such a plot with different types of peripheral arm compliance. As illustrates the horizontal arm is the central point and transmural changes are evaluated relative to this reference. Stiffer vascular system results in a higher slope while more compliant system have a lesser slope. The transmural pressure changes as well as the changes in pulse wave velocity are from a resting or initial state. Specifically, no blood pressure measurement is required to generate FIG. 22 since calculation or slope determination is relative to a resting condition. The resting condition can be, but is not limited to, arm down or arm horizontal.

Central Compliance Curve

Unlike the determination of peripheral compliance where an isolated transmural pressure change can be created by using an arm raise, transmural pressure changes in the central cavity can be achieved using resistance breathing, which alters transmural pressure around the thoracic aorta and stroke volume.

Determination of Central Compliance Curve

Changes in stroke volume can be used to generate changes in central compartment transmural pressure. The larger the stroke volume or the amount of blood pushed out by the heart with a contraction, the larger the volume of blood moving through the central arteries and the higher the transmural pressure. Changes in intrathoracic pressure impact venous return to the heart which impacts stroke volume and pulse pressure which impacts pulse wave velocity. The physiologically relationships between intrathoracic pressure, volume status, stroke volume and changes in systolic and pulse pressures is been well studied during mechanical ventilation. These relationships are well described by Frederic Michard in the publication, “Changes in arterial pressure during mechanical ventilation.” The Journal of the American Society of Anesthesiologists 103.2 (2005): 419-428. Warltier, D. C., & Ph, D. (2005). Changes in Arterial Pressure during Mechanical, (2), 34-36. It is important to note that these changes were observed during mechanical ventilation.

The use of mechanical ventilation is not practical for a simple screening test but a method for increasing stroke volume changes and creating transmural pressure changes is to change intrathoracic pressure via resistance breathing. Controlled breathing or resistance breathing is the process of increasing, decreasing, or both increasing and decreasing the magnitude of pressure needed to exhale or inhale. The result is a more dramatic change in intrathoracic pressure and larger variances in stroke volume.

Embodiments of the current invention use controlled breathing to create repeatable intrathoracic perturbations. The process does not include mechanical ventilation and is distinguished from common spontaneous breathing in that the breathing activity is volitional. Controlled breathing represents a volitional activity of the patient and includes properties of pace (or rate) as well as pressure. The result is a systematic perturbation that changes intrathoracic pressure in a defined and repeatable manner.

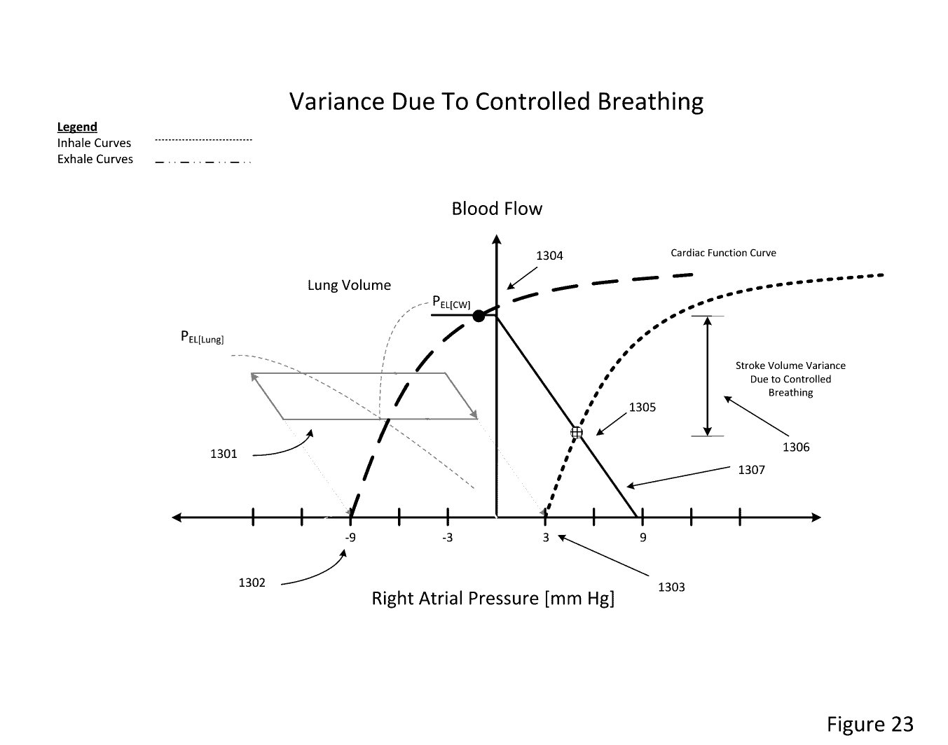

The value of a controlled breathing process can be well illustrated through use of the Combined Heart-Lung diagram. This process is diagrammed in FIG. 23 with a −6 and +6 mm Hg controlled breathing protocol. Note the “flat” or “box” portion of the Campbell diagram shows the influence of the resistance threshold system. The pressure increases with little change in lung volume until the threshold of the device is obtained. The device then maintains a moderately constant pressure until the exhale or inhale is completed, see 1301 as an example of “flat portion” of inhalation. Note also the large left shift of the cardiac function curve with inhalation, 1302, and the opposite right shift of the cardiac function curve with exhalation, 1303. These changes impact the cardiac operating points as shown in 1304 for inhale and 1305 for exhale. The resulting cardiac operating points cause in a large change in the cardiac output or stroke volume. The change is identified by arrow 1306 which shows the difference in cardiac output between the inhale and exhale. 1307 shows the venous return curve in this schematic. The stroke volume change 1306 creates a transmural perturbation that can be used generation of a central compliance curve.

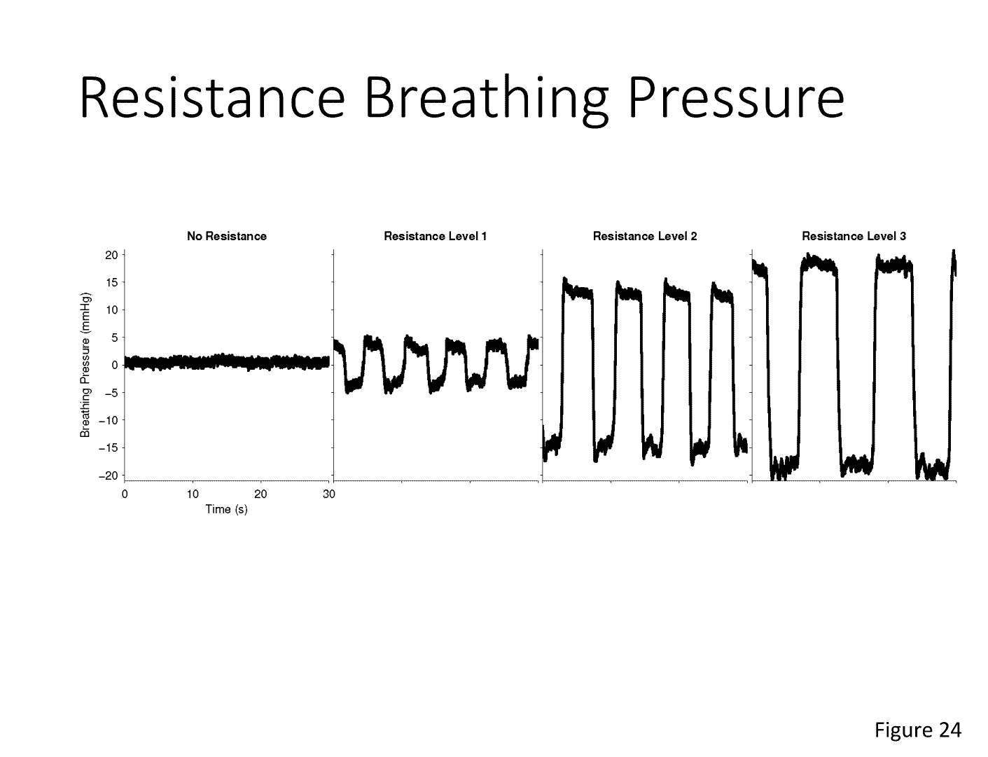

The controlled breathing system can be configured so that pressures are the same on inhalation and exhalation (symmetric) or different on inhalation and exhalation (asymmetric). FIG. 24 shows and example of increasing resistance breathing. Note that the resistance pressure can be modified to facilitate different defined intrathoracic pressure changes. The resistance pressures can be used to magnify normal changes in intrathoracic pressure leading to larger changes in venous return thus effectively creating a measurable change in stroke volume and arterial pressure.

In testing, most subjects exhibit a higher degree of comfort with variable exhalation testing. Thus, the following use scenario can be envisioned. The device is provided to the patient, and PPG signals of sufficient quality are confirmed. The subject begins breathing at a defined rate of 6 breaths per minute. The subject continues to execute the breathing protocol until a constant breathing pattern is obtained as assessed by breath timing and air flow characteristics. Based upon testing, most individuals need time to get comfortable with the system. The system can provide feedback to the user as needed. The base condition has a low level of resistance at 2 cm H20 on both inhalation and exhalation. Following procurement of a consistent breathing profile, the system adds some additional exhalation resistance in a slow and systematic manner. Resistance can be added at a rate of 5 cm H20 per minute or at a rate of 5 cm H20 per 6 breaths. The result is a 3-minute test that creates continuous curve of changing intrathoracic pressure with a maximum exhale pressure of 15 cm H20. If instabilities in the measurements are observed, the system can prompt the subject to repeat the measurement/breath at the prior pressure.

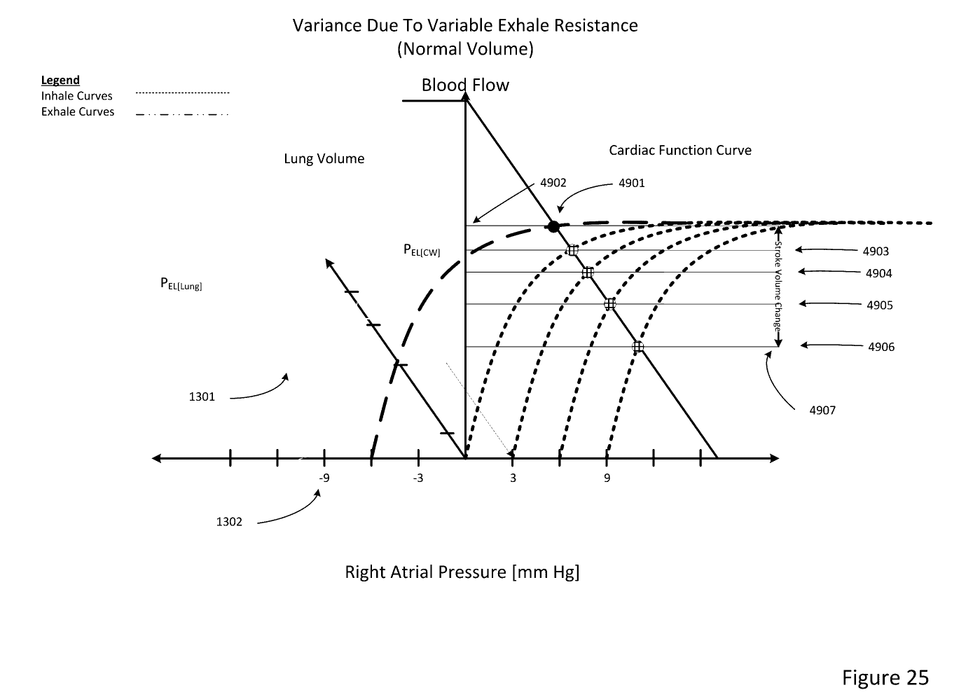

The value of the above method, in addition to patient convenience, can be shown via combined heart-lung curves. FIG. 25 is a combined heart-lung graph showing variable exhalation pressure under a condition of normal volume. 4901 represents the cardiac operating point for the inhale condition which remains fixed over the test. The resulting cardiac output is shown on the y-axis as point 4902. Line 4903 shows the cardiac output during the first exhale pressure. 4904 is the second exhale pressure, 4905 the third and 4906 the fourth. The resulting change in stroke volume is illustrated by arrow 4907. With increasing exhalation pressure, the change in stroke volume increases. This systematic change creates the change in transmural pressure needs for development of a compliance curve.

The changes in stroke volume will result in arterial pressure changes. These beat-to-beat pressure changes in combination with pulse wave velocity measurements enable the generation of central compliance curve.

As the intention of the system is to measure central compliance, the measurement is facilitated by measuring pulse wave velocity across the aorta. Thus, the PPG measurement sites should be located such that the pulses have transverse central compartment.

The determination of beat-to-beat blood pressure or measurements that are indicative of blood pressure changes can be accomplished using several different methods. Continuous noninvasive arterial pressure measurements on a beat-to-beat basis can be obtained using the methods developed by Czech physiologist Jan Pe\check{n}áz. Additionally, the change in arterial pressure are proportional to change in stroke volume which is proportional the left ventricular ejection times. LVET can be determined from PPG pulse waveforms recorded at peripheral sites such as the finger. An estimate of blood pressure variance can be obtained by measurement or determination of the intrathoracic pressure change. The resistance breathing deice has a threshold pressure needed such that air movement occurs. This intrathoracic pressure influences venous return has an indirect influence on the observed changes in arterial pressure.

The resulting pulse wave velocity information in conjunction with information on arterial pressure can be used in the same manner as the peripheral information to create a central compliance curve.

Demonstration of Central Compliance Measurement Curve

A demonstration of the method was achieved by placing a subject in a seated position with PPG sensors attached to the subject's toe and finger. The subject rested quietly for several minutes followed by a controlled breathing protocol. The paced breathing protocol consisted of exhalation and inhalation resistance of 20 mmHg, with an exhalation period of 10 seconds and inhalation period of 6 seconds. Figure shows the physiological impacts of the resistance breathing. The plot shows the measured pressure at the resistance breathing device (which approximates intra-thoracic pressure), the change in stroke volume, the change in arterial pressure, and the change in pulse wave velocity. Figure shows the relationship between aortic transmural pressure and pulse wave velocity for the test performed. Aortic transmural pressure (TMP) is computed as TMP=MAP−ITP, where MAP is the mean arterial pressure and ITP is the intra-thoracic pressure. The slope of the line between MAP and PWV is indicative of arterial compliance, with larger slopes indicating greater compliance.

As one of ordinary skill will appreciate, there exist other mechanisms for creating changes in transmural pressure and stroke volume that include but are not limited to mechanical ventilation and hydrostatic positional changes. Hydrostatic positional change is a general term that applies to any process that changes the hydrostatic pressure in a vessel due to positional changes and include passive lower leg raises, head tilts, standing up, movement form the supine to sitting position, etc. Compensation of hydrostatic pressure changes in the central compartment can be corrected for by determination of sensor positions relative to each other and relative to the heart.

Additional Central Compliance Considerations

Redistribution of blood volume by positional changes can also be used to assess central volume compliance. For example, the process of raising the lower legs when a patient is in the supine position transfers approximately 150 mL to the central cavity and increases the mean circulatory pressure. (Monnet, Xavier, et al. “Passive leg raising predicts fluid responsiveness in the critically ill.” Critical care medicine 34.5 (2006): 1402-1407.). If lower leg elevation were to be used the PPG measurement site could be relocated to the upper tight or any skin site that does not undergo significant elevation changes, is supplied by blood traveling through a significant portion of the aorta.

Improved Compliance Measurement

The relationship between pressure and compliance (or distensibility) is highly nonlinear. With increasing pressure, the vascular system becomes less compliant. Figure shows the relationship between distensibility and pressure for a group of subjects ages 30 to 88. The data are from the Langenwouters 1984 paper. By examination of this graph, differences in distensibility are most easily determined at lower vascular pressures. As the vascular pressure increases, there is an asymptotic progression to a common value. Therefore, at high pressures such as those in box 800, the differences in distensibility are small. However, examination at pressures such as shown in box 802 show moderately significant differences. Embodiments of the invention can determine both central and peripheral compliance; actions that reduce vascular pressure can facilitate the overall sensitivity of such determination.

As it relates to measurement of central compliance, reduced vascular pressure can be achieved immediately after standing as there is distension of the venous system in the legs, which corresponds to a rapid accumulation of 300 to 800 ml of blood in the legs and a lower venous return. Other positional changes can be used to redistribute blood volume. In addition to positional changes, exhalation resistance breathing or the Valsalva maneuver can be used to increase intrathoracic pressure. The increase in intrathoracic pressure results in both decreased venous return to the heart as well as increased transmural pressure. The actual physiological response to the Valsalva maneuver is complex but the transient decrease in blood pressure is approximately 20 mmHg, see Figure.

An important benefit of some embodiments of the invention is the ability to acquire measures of arterial stiffness at blood pressures that are below standard physiology for improved sensitivity associated with arterial stiffness measurements. As shown in Figure, changes of as little as 10 mmHg can improve the sensitivity of the measurement appreciably. The measurement of arterial stiffness under conditions of reduced vascular pressure are presented in the example embodiments.

Improved Processing Methods

Use of Ancillary Information

Arterial compliance as described above is influenced by the development of diabetes and there is a strong relationship between aortic compliance and deteriorating glucose metabolism status. Additional factors can also influence arterial compliance. A well-recognized change in arterial compliance occurs with age. Figure shows regression curves showing the effect on age on pulse wave velocity for males (circles, solid lines) and females (squares, dashed lines), from McEniery C M, Yasmin, Hall I R, et al. Normal vascular aging: differential effects on wave reflection and aortic pulse wave velocity: the Anglo-Cardiff Collaborative Trial (ACCT). J Am Coll Cardiol 2005, 46:1753-1760.) Performance improvements can be obtained by effectively normalizing out or compensating for known influences of arterial compliance. In very simple terms, the diagnostic process can utilize age as an input variable and effectively compensate for the normal aging process.

To implement a high-performance screening device, additional ancillary information can be utilized by algorithms to normalize, compensate, or adjust for these known influences. Example candidate ancillary parameters include but are not limited to age, hypertension, duration of hypertension, height, weight, waist size, size, heart rate, mean arterial pressure, systolic pressure, diastolic pressure, creatinine, smoking, hypertensive medications, cholesterol, low-density lipoproteins, high density lipoproteins, albumin, plasma homocysteine, smoking duration, triglycerides, alcohol consumption, ethnicity, C-reactive protein, gender, hemoglobin, hematocrit and urea.

In use, these pieces of ancillary information can be entered at the time of use, accessed from an electronic medical record, or in some other manner introduced into the algorithm for appropriate consideration. These variables can also be used to adjust a numerical output.

Pulse Wave Contour Analysis

Pulse wave velocity is the most common metric for determining arterial stiffness. However, several other approaches exist that quantify other elements of the arterial pulse wave and are broadly classified here as pulse wave analysis. Elgendi et al. provide a reasonable list of metrics that can be calculated from a pulse wave (Elgendi, M. (2012). Standard Terminologies for Photoplethysmogram Signals. Current Cardiology Reviews, 8(3), 215-219.). The above reference paper is incorporated by reference. Analysis of the aortic pressure waveform provides a measure of central blood pressure and indices of systemic arterial stiffness, such as Augmentation Pressure (AP) and Augmentation Index (AIx). These parameters are rather simplistic methods that use peak heights or ratios of peak heights for the determination of various parameters. Figure shows a typical method for the calculation of augmentation index. In the figure: central aortic waveform and augmentation index (AIx); (A) forward wave; (B) reflected waveform; (c) summation waveform as the result of early wave reflection in a patient with stiff arteries.

Demonstration of Improved Pulse Wave Contour Analysis

In addition to pulse wave velocity assessments, significant additional information regarding aortic compliance is available through analysis of the amplitude of the wave, the frequency components of the wave, and the overall shape of the wave. Figure shows the change in contour and amplitude of pressure waves recorded in the radial artery in normal subjects between the first and eight decades of life. These age-related changes in pulse contour shape are due to increasing stiffness of the vasculature with age.

As stated previously, prediabetes and diabetes lead to accelerated aging of the vascular system. Contour or shape-based methods based can be used to determine the “effective age” of a recorded pulse profile. For example, if a 40-year-old individual were to have a pulse waveform more consistent with that of a 60-year-old individual it can be indicative of significant arterial aging and compliance changes.

In “Non-invasive estimate of blood glucose and blood pressure from a photoplethysmograph by means of machine learning techniques”, Monte-Moreno, Enric, Artificial Intelligence in Medicine, Volume 53, Issue 2, 127-138, the authors use a number of techniques to clean, filter, and extract features from photoplethysmographs. The various filter methods applied to the PPG signal create a number of different metrics. For example, energy, Qi-Zeng energy, and entropy crossing rate are computed by a FFT transform of the PPG signal. All computed quantities are collected into a vector of features that is used to train several classification approaches (Linear mode, Neural Networks, Support Vector Machines, Classification and Regression Trees and Random Forest) to determine blood pressure or blood glucose. The method is not used for determination of pulse wave velocity, arterial compliance, or any assessment of diabetes state.

In “Multi-Gaussian fitting for pulse waveform using Weighted Least Squares and multi-criteria decision making method”, Wang, Lu et al., Computers in Biology and Medicine, Volume 43, Issue 11, 1661-1672, authors use well known techniques of fitting a number of Gaussian curves to represent photoplethysmograph signals. The approach decomposes the physiological signal generated from an appropriate instrument into a number of Gaussian curves. The sum of Gaussian curves is fitted to the physiological curve by mean of Weighted Least Squares. Goodness-of-fit is estimated and studied in the paper. The paper provides a mechanism for fitting pulse waveforms but does not articulate a use for the fitted parameters. The approach is limited by assuming that the signal is composed of only Gaussian curves. Thus, no relationship is defined between these Gaussian curve fits and the desired measurement parameter of arterial stiffness or diabetes-hypertensive state.

In “Arterial stiffness estimation based photoplethysmographic pulse wave analysis”, Matti Huotari et al., Proc. SPIE 7376, Laser Applications in Life Sciences, 73760L (Nov. 24, 2010), authors used signals generated from photoplethysmograph devices, and analyzed them by decomposing the signal into a small number (five) of component functions fitted by non-linear least square minimization with the Levenberg-Marquart approach. The fitting errors shown in the publication, FIGS. 3, 4 and 5 show significant residual error, especially during the systolic phase. Despite the publication's title, no true relationship is shown between measured parameters and arterial stiffness. The paper correctly assumes some relationship with age and arterial stiffness, and does show general trends associated with age and the calculated parameters. Again, no direct measure of arterial stiffness is presented and no association with diabetes is articulated.

In “Radial pulse transit time is an index of arterial stiffness”, Zhang, Yong-Liang et al., (Hypertens Res, 2011, Volume 34, Number 7), use pulse pressure data obtained by an applanation tonometer based system and process the data to determine the arrive of the first and second systolic peaks. The resulting time difference or Pulse Transit Time (PTT) is used to create a time difference that is correlated with age. The authors infer a general trend between arterial stiffness and age and show a correlation between the time difference and age. The method is limited to only a peak detection method and no direct measure of arterial stiffness is presented and no association with diabetes is articulated.

The present invention can address the limitations of the prior art with a focus on diabetes assessment and hypertension assessment by the effective use of pulse transit time, use of reflected wave information, heart rate variability, pulse amplitude, frequency content determination, and the shape of the pulse wave. The present invention is not constrained by historical limitations that compromise the degree of fit, make assumptions regarding curve shape (for example Gaussian) or use only peak separation metrics. These limitations limit the information that can be obtained for the pulse wave by ignoring higher order harmonics or failing to use effective curve parameterization techniques.

The feature vector used can be derived from a singular observation or a series of observations. Specifically, the vector of features can include pulse data obtained under different conditions such as arm down/up, or during resistance breathing, or changes in position. These activities create feature vectors with different pressure or transmural conditions. Additionally, the feature vector can include heart rate information, autonomic information, and autonomic response information. The feature vector can include multiple PPG data signal from different locations on the body including finger to finger pulse agreement information.

The use of current technology machine learning techniques can provide superior results to historical approaches. The raw PPG signal can be processed or decomposed using multiple methods including but not limited to discrete wavelet transform (DWT), fast Fourier transform (FFT), individual component analysis (ICA), t-distributed stochastic neighbor embedding techniques and other related methods. The resulting information can be processed by multiple classification or machine learning techniques including but not limited to random forest classifiers, partial least squares, deep learning methods, tensor flow techniques, support vector machines, decision trees or any type of classifying trees, clustering, Bayesian networks, neural networks, etc. The resulting information can be provided to the clinician as a diabetes or hypertension assessment with confidence interval information.

To demonstrate the value of a contour analysis approach an example using simulated data was created. The data used in this example was simulated based upon an extensive literature review of the contour changes that occur due to aging and diabetes. The resulting vector of features is then processed or evaluated by a classifier developed by one or more machine learning/pattern recognition approaches. The output of the classifier defines a metric associated with diabetes state or an assessment of diabetes.

To demonstrate the superiority of the method, a set of simulated data modeled after representative physiological signals was generated and subsequently analyzed according to the methods described herein. 2,000 photoplethysmographs from subjects at various stages of Diabetes Mellitus (but without other concomitant co-morbidites) and 2,000 photoplethysmographs from otherwise similar healthy subjects were simulated. Figure shows an example of the pulses used for analysis. The data represents a complex and confusing array of pulses with waveform differences. The resulting PPG signals are decomposed by mean of Discrete Wavelet Transform (DWT). The resulting wavelet coefficients are extracted to form a feature vector. One feature vector was created for each case subject. The wavelet feature vector was used to train a support vector machine classifier. Subjects were randomly assigned to the training and test sets. The classifier was trained using the training set only. The test or validation subjects were then classified by the Support Vector Machine classifier and the performance was accessed by Receiver Operation Characteristic (ROC) curve. As a control and to compare the goodness of the improved approach, Pulse Transit Time (PTT) as used by Zhang, Yong-Liang et al. was computed on of the very same dataset. Zhang et al calculated pulse transit time (PTT) by the time interval between the first and second peaks of the radial pulse wave, not the standard method.

Examination of Figure shows the significant performance improvement possible using a multivariate machine learning approach, rather than a univariate approach. The Area Under Curve (AUC) for the Wavelet approach is greater than the AUC for the PTT approach and the test performance is better at every point on the ROC curve. The performance with a false positive rate of 25% is a sensitivity of 85% (240) for the Wavelet approach in comparison to a 68% (241) for the PPT approach. The improved processing method yields a 25% increase in sensitivity. Machine learning with wavelet feature decomposition has demonstrated superior diagnostic power relative to historical pulse transit time based approaches.

Improved Autonomic Testing Method

Autonomic System Reaction Time.

The autonomic nervous system is responsible for maintaining blood pressure. For example, as you stand up, the autonomic nervous system will rapidly adapt to the changes in volume distribution. Historical work in this area has shown that the transition from a supine position to a standing position will result in a maximum heart rate at around the 15^{th} beat. The work by Ewing et al. examined this phenomenon between subject groups of young and old controls versus diabetics with and without neuropathy (1. Ewing D J, Campbell I W, Murray a, Neilson J M, Clarke B F. Immediate heart-rate response to standing: simple test for autonomic neuropathy in diabetes. Br Med J. 1978; 1(6106):145-147.). The autonomic nervous system response between these subject classes was different. The arm location changes used for determination of peripheral compliance as well as the transmural pressure changes used for determination of central compliance will initiate an autonomic nervous system reflex. The overall response of this reflex, including the shape of the response, the heart rate intervals, duration of response, the magnitude of the response, and other parameters will be highly diagnostic of the condition of the autonomic nervous system. All of the above pressure changes represent a stress test of the autonomic nervous system with a corresponding response. In general terms, stress tests are typically more sensitive than static tests for assessing functionality.

An illustration of autonomic testing is to examine the level of agreement between the PPG signal obtained from the two arms during the stress testing. In an individual with normal autonomic function, the correlation between the two pulse waves in the finger will exhibit excellent correlation during a bi-lateral arm raise. In the presence of autonomic dysfunction, level of agreement or correlation decreases. The physiological reaction to this perturbation is very important since it represents a time response component, a magnitude of response component, and a shape response component. These parameters can be used to accurately access automatic function and improve the overall diagnostic value of the system.

Another illustration of autonomic testing is to examine the relationship between the cardiac beat-to-beat interval (RR) and PTT or PAT, using resistance breathing to generate changes in both variables. In a subject without diabetes, heart rate variability is high during resistance breathing and there exists a defined relationship between changes RR interval and PPT. In the patient with autonomic dysfunction, the variability will be reduced.

Signal-to-Noise Enhancement and Integrated Model

Diabetes and hypertension are disease conditions that are not defined by a singular physiological change but by overall alterations that deviate from normal physiology. For example, diabetes is often considered to be the inability to regulate glucose to normal levels, but in fact diabetes is a combination of many physiological changes. As described above, diabetes for example causes changes in vascular stiffness, autonomic function, and microvascular changes to name a few. Therefore, the ability to effectively incorporate all sources of information effectively for the highest performing test is an objective of this invention. The ability to combine multiple sources of information results in an effective signal-to-noise improvement of the test. The diabetes assessment or hypertension assessment can be determined through state-of-the-art multivariate modeling techniques or machine learning techniques. In this manner, the assessment score is based upon a feature vector of information provided to the algorithms. Elements of the feature vector can include but are not limited to age, gender, pulse rate, heart rate variability, mean blood pressure, pulse wave velocity, aortic transit time, autonomic function characterization, right versus left PPG agreement, arm swing compliance information, central compliance information, and all other parameters typically use to describe characteristics of a pulse wave. As mentioned previously, Elgendi defines these parameters effectively in his 2012 paper. It is important to note that these parameters will vary due to heart rate, respiration, as well as specific perturbations such as resistance breathing, standing, or lower leg raises. Therefore, the feature vector may include average determinations or information that provides effective representation of variances. For example, the variance representation may include but is not limited to spread, symmetry, skew, dispersion, range, and other statistical assessment. Additionally, many of the parameters measured are repetitive in nature and highly amenable to frequency analysis including Fourier transform and wavelet analysis. The resulting feature vector can be used to train both regression-based multivariate algorithms as well as classification based algorithms. These types of feature vectors are amenable to decision trees including random forests and other such techniques.

Example Embodiment and Method

The method and system described herein create a remarkably simple test that provides information associated with both vascular stiffness as well as autonomic function. The device shown in Figure includes a hand based EKG measurement system (711), a right and left finger PPG measurement system (710), display and inertial measurement unit (inside device). Information displayed to the patient is shown in Figure, where demographic, physiological and operational information are displayed. Operational information including feedback on breathing (801) as well as arm location (802) is provided to the patient. An example measurement protocol is as follows:

Enter subject information such as age, gender, height and weight, see Figure

Acquire a standard brachial blood pressure

Sit patient on an examination table and have them hold the device

Attach PPG clips (701) to left and right fingers, as shown in Figure.

Obtain a baseline measurement of heart rate, and PPG pulses from both fingers while the subject holds the device with their arms down, as shown in Figure, and labeled as position 0 degrees. A PPPG sensor for measurement of PPG signals after traveling though the aorta can be attached to the ankle as illustrated by 3701

The device display may provide verbal or graphical instruction regarding paced breathing, see Figure, (801) as the arm location is noted by the dark filled-in arm location.

Following the baseline measurement period, the device will instruct this subject to keep the arms straight and too slowly raise the device above the head.

The inertial measurement system (IMU) has the ability to determine the device's velocity and orientation and will provide feedback if the subject is going too fast, too slow or not executing the movement correctly.

Depending upon the protocol, the system may request that the subject stop their arm movement at defined angular locations, such as 45°, 90°, 135° and 180°. The rotation of the arm above heart causes a change in transmural pressure due to hydrostatic pressure. In addition to changes in hydrostatic pressure, the elevation of the arm will create an autonomic reflex that can be of significant diagnostic value. Figure shows an example of three position and approximately 0, 90 and 180 degrees.

The amount of time spent at each position may vary depending upon the response time of the autonomic reflex.

The device may request the subject reverse the motion, lowering the device back to the starting point.

If the subject does not execute the movements correctly, the device will inform the subject and will repeat the measurement protocol until movements are performed satisfactorily.

Following completion of this initial phase of the test, the device will be returned to its starting position at rest on the thighs.

Following a brief rest for the subject, the second phase of testing involving resistance breathing will be initiated, see Figure. 901 is an example of a resistance breathing device. The device can take a variety of forms depending upon the exact resistance breathing protocol defined. A mask or mouth piece system can be used.

The subject will breathe through a resistance breathing device (901) that creates one or more changes in intrathoracic pressure. As previously noted the protocol may include but is not limited to one of the following: inhalation resistance, exhalation resistance, or both.

The changes in intrathoracic pressure will also cause changes in autonomic function that will be measured by the system. The above data can be acquired in a continuous fashion or at incremental steps. The resulting information can be used to assess peripheral compliance, central compliance, and autonomic function for determination of diabetes and hypertension assessment. The specific information determined includes but is not limited to:

Heart rate variability

Peripheral arterial compliance information

Central compliance information

Autonomic response time to various perturbations

Correlation between heart rate changes and breathing

Relationship between right and left finger pulses

Systolic and diastolic blood pressure

Length of arm is determined by the inertial measurement unit

Length of torso as determined by the inertial measurement unit

The described method and device enables the effective assessment of both vascular status and autonomic nervous system function. System algorithms can process the above information to determine overall diabetes status as well as the likelihood of hypertension.

Figure shows an example of how peripheral and central compliance can be used to diagnose the patient. As shown in the figure, subjects with peripheral and central compliance values in the upper right are considered normal, without evidence of diabetes or hypertension. The dashed lines indicate that the definition of normal by central and peripheral compliance may need to be adjusted by the age of the subject, as it well recognized that the vascular system becomes stiffer with age. The lower right corner is defined as diabetic because individuals with diabetes have decreased central compliance with less changes in the peripheral vascular system. The lower left is for subjects with low compliance for multiple reasons including hypertension and hypertension with diabetes. Diabetes and hypertension are considered additive as it relates to decreasing compliance. Figure is for general illustrative purposes and solely intended to explain the value of independent measures of central and peripheral compliance. It is also important to note that autonomic function assessment and pulse wave contour information can be included to further improve diagnostic resolution. These important pieces of additional information are not picture on the illustration.

The inclusion of blood pressure within the decision matrix provides additional value as shown in Figure. The diagnostic criteria are similar to those in Figure but the decision matrix allows for the determination of White Coat Syndrome. An individual with White Coat Syndrome may have an elevated blood pressure but none of the associated vascular stiffness associated with hypertension. Specifically, the subject has age-adjusted normal compliance for both central and peripheral arteries.

The above decision information can be translated into a singular metrics such as a Diabetes Assessment Score (DAS) which is reported on a scale of 0 to 100. The higher the DAS value, the more likely the subject has type 2 diabetes. Subjects with DAS results 50 are considered a positive screen for pre-diabetes or type 2 diabetes and should have a follow-up blood test to make a diagnosis. As one of skill in the art will appreciate, the DAS can indicate the presence or likelihood of diabetes; the degree of progression of diabetes; a change in the presence, likelihood, or progression of diabetes; a probability of having, not having, developing, or not developing diabetes; the presence, absence, progression, or likelihood of complications from diabetes. “Diabetes” includes a number of blood glucose regulation conditions, including Type I, Type II, and gestational diabetes, other types of diabetes as recognized by the American Diabetes Association (See ADA Committee Report, Diabetes Care, 2003), hyperglycemia, impaired fasting glucose, impaired glucose tolerance, and pre-diabetes.

As it relates to hypertension, a similar metric can be generated. The Hypertension Assessment Score (HAS) would also be reported on a scale of 0 to 100 or with gradations of severity. The higher the HAS value, the more likely the subject has hypertension. Subjects with HAS results 50 are considered to have a positive screen for hypertension and should have a follow-up with additional testing.

### Second Example Embodiment

A second example embodiment of the system is shown in Figure. This example system utilizes a PPG measurement device located at the ear or forehead (1001) and finger (1002) with an ECG measurement system on the chest (not shown and optional in some measurement scenarios). The overall method of operation is similar to that previously presented but only one arm is utilized to generate the peripheral and central compliance assessments. The processing of the data also differs since the ECG and forehead based PPG information can be utilized to capture a pulse transit time that is preferentially specific for the central vascular system. Additionally, the pulse transit time as measured with the forehead PPG and the finger PPG is quite specific for arm transit time. The resulting information enables assessment of central and peripheral compliance as well as autonomic response. These measured parameters can be used to screen for both diabetes and hypertension.

The embodiment shown in Figure can also utilize different wavelengths for obtaining the PPG signal. For example, the forehead sensor may use a wavelength with high hemoglobin absorbance such as is in the 500 to 650 nm range, while the transmission wavelength might be in the 800 to 950 nm range. Additionally, the system may provide a direct hydrostatic pressure assessment by having a single tube of water where the ends are attached at the heart and the finger (not shown). Such a system can be used to obtain a direct measure of hydrostatic pressure.

As one of ordinary skill in the art will appreciate, there are numerous variations possible based on the above systems.

Operation of System

In practice, embodiments of the present invention create a method and system for diabetes and hypertension assessment that is remarkably easy to use. The test does not have a fasting requirement, because the test is not a direct assessment of point in time glucose maintenance. In contrast, the measurement effectively integrates the damage due to slight variations in glucose as well as the early manifestations of diabetes by examination of vascular stiffness and autonomic nervous system function. The system also enables hypertension screening using similar methods with or without a blood pressure measurement. Therefore, any individual can be tested at any time without pretest fasting issues. The individual under examination can simply hold the device for a period of time to enable the device to obtain data during at least one cardiac cycle. A diabetes assessment score and hypertension assessment score can be provided to the patient at the time of testing which is extremely valuable in terms of follow-up testing and immediate counseling. The numerical value of the risk score can provide the individual with information regarding their probability of having prediabetes or diabetes as well as hypertension. Additionally, the device can provide a more direct assessment by simply defining an individual state as normal, pre-diabetic, or diabetic. The device can also, or as an alternative, provide an “arterial age” measurement. Such measurement can simply state that the information obtained from the patient is most consistent with a given age profile. Accelerated aging would be viewed as problematic and additional medical work required.

Additional Optical Systems.

As one of skill in the art will recognize, there are multiple instrument variations that can be used for the collection of data. For example, the calculation of pulse transit time or other measures of arterial stiffness can be obtained from a simple conventional oximeter using standard LEDs or a PPG specific measurement system.

Additional Pulse Measurement Systems:

The current disclosures focused on the use of optical measurement systems for the determination of pulse waves. Any system that effectively records the pulse wave can be utilized in the proposed system. Such devices can include, but are not limited to, pulse pressure transducers, applanation tomography systems, and ultrasound systems. Oscillometric measurement devices are especially applicable because they create high fidelity waveforms. The operation of an example device is well covered in a paper by Wassertheurer et al, (Wassertheurer, S., Kropf, J., Weber, T., van der Giet, M., Baulmann, J., Ammer, M., . . . Magometschnigg, D. (2010). A new oscillometric method for pulse wave analysis: comparison with a common tonometric method. Journal of Human Hypertension, 24(8), 498-504). The operation of such systems varies significantly, and various systems utilize different pressures for recording waveform measurements. These devices can be adapted to the finger or other locations such that high-resolution pulse waveforms can be obtained. These devices can be utilized in conjunction with this invention for the effective development of a diabetes screening system. Ultrasound can also be used to measure the arrival of pulses as well as pulse wave velocity. One of ordinary skill can appreciate the substitution of ultrasound or sound based pulse measurement methodologies into the current invention for diabetes and hypertension assessment.

The present invention has been described in the context of various example embodiments. It will be understood that the above description is merely illustrative of the applications of the principles of the present invention, the scope of which is to be determined by the claims viewed in light of the specification. Other variants and modifications of the invention will be apparent to those of skill in the art.

We claim:

1. A method to assess vascular stiffness of a subject, comprising determining arrival at a peripheral site of a blood pressure wave as a function of time relative to the cardiac cycle of the subject at a plurality of measurement conditions, wherein at least two of the conditions are characterized by at least one of: (a) different central transmural pressure, (b) different peripheral transmural pressure; assessing vascular stiffness from the determinations at the plurality of measurement conditions.

2. A method as in claim 2, wherein the plurality of measurement conditions are characterized by one or more of: resistance breathing, intrathoracic pressure, positional changes of the subject, hydrostatic pressure changes, peripheral vascular location, external pressure on the peripheral vasculature.

3. A method of assessing diabetes comprising assessing vascular stiffness according to claim 2, and assessing diabetes from the assessment of vascular stiffness.

4. A method of assessing hypertension comprising assessing vascular stiffness according to claim 2, and assessing hypertension from the assessment of vascular stiffness.
